(19) United States
(12) Patent Application Publication
(10) Pub. No.: US 2012/0219613 A1
(43) Pub. Date: Aug. 30, 2012

(54) Novel Antiviral Agents for Enveloped Viruses

(75) Inventors: Benhur Lee, Los Angeles, CA (US); Michael E. Jung, Los Angeles, CA (US); Michael C. Wolf, Los Angeles, CA (US); Tinghu Zhang, Los Angeles, CA (US)

(73) Assignee: THE REGENTS OF THE UNIVERSITY OF CALIFORNIA, Oakland, CA (US)

(21) Appl. No.: 13/000,310

(22) PCT Filed: Jun. 18, 2009
(86) PCT No.: PCT/US09/47854

§ 371 Date: Apr. 26, 2011

(30) Foreign Application Priority Data
Mar. 23, 2009 (US) 61162486

(51) Int. Cl. A61K 31/427; A61P 31/18; C07D 495/04; A61P 31/16; A61K 9/127; C07D 417/06; A61P 31/14; A61P 31/22

(52) U.S. Cl. 424/450; 514/369; 548/183

(57) ABSTRACT

Provided herein are a series of arylmethylidene rhodanine derivatives having broad-spectrum antiviral activity against enveloped viruses, including but not limited to filoviruses, poxviruses, arenaviruses, bunyaviruses, paramyxoviruses, flaviviruses, influenza A, and HIV-1. The compounds act via a novel mechanism to disrupt viral membranes and inhibit viral attachment, fusion, and/or entry into host cells. The membrane disrupting activity of the compounds is selective for viral membranes relative to other biological membranes, making the compounds non-toxic to cells and host subjects.

### CROSS-REFERENCE TO RELATED APPLICATION

The present application claims the benefit of U.S. Provisional Patent Application No. 61/073,448, filed Jun. 18, 2008, and U.S. Provisional Patent Application No. 61/162,486, filed Mar. 23, 2009, both of which are herein incorporated by reference in their entirety.

### STATEMENT OF GOVERNMENT SUPPORT

This invention was made with United States Government support under National Institutes of Health Grant No. AI070495. The United States Government has certain rights in this invention.

### BACKGROUND

There are few licensed and efficacious broad-spectrum antivirals currently available. Examples include ribavirin, which functions via nebulous effects on both host and virus proteins, and alpha-interferon, which produces unwanted side effects and remains impractically expensive for widespread use (Tam et al., Antivir Chem Chemother, 12:261-72 (2001); Bekisz et al., Growth Factors, 22:243-51 (2004); de Veer et al., J Leukoc Biol, 69:912-20 (2001); Sen, Annu Rev Microbiol, 55:255-81 (2001); Hong and Cameron, Prog Drug Res, 59:41-69 (2002)). The prevailing paradigm in antiviral research emphasizes a “one bug-one drug” strategy; however, the rapid rise in the number of emerging viral pathogens brings into stark contrast the limited resources available to develop therapeutics on a single-pathogen basis (see e.g., Burroughs et al., “The Emergence of Zoonotic Diseases: Understanding the Impact on Animal and Human Health,” Workshop Summary from Board on Global Health, Institute of Medicine, National Academy Press, Washington, D.C. (2002)). The expense and difficulty of developing antiviral drugs tailored to specific pathogens underscores the need to develop broad spectrum antiviral drugs against targets that are common among large classes of viruses.

Viruses can be categorized as either lipid-enveloped or non-enveloped (naked). Enveloped viruses replicate within the host-cell, recruit viral proteins to the host membrane, and then bud from and utilize the host membrane, essentially, as a vehicle to transport the viral genome to new cellular targets. Although the lipid membrane of enveloped viruses derives from the host cell, it differs from host cellular membranes in several biochemical and biophysical properties, such as biogenic reparative capacity, fluidity, lipid composition, and curvature. For example, the membranes of budding viral particles are highly curved relative to the membranes of much larger host cells. As a result, the fusion of enveloped viral particles with new host cells requires that the high curvature viral membranes undergo elastic stresses and subsequent negative curvature needed to promote fusion between the juxtaposed outer lipid monolayers of the viral particles and host cell membranes (Chernomordik et al., J Cell Biol, 175:201-7 (2006); McMahon and Gallop, Nature, 438:590-6 (2005); Chernomordik and Kozlov, Annu Rev Biochem, 72:175-207 (2003)).

The central role of virus-host cell fusion in the infectivity of enveloped viruses has motivated the development of small molecule antiviral therapeutics that insert, intercalate, or otherwise bind to viral membranes and disturb the membrane dynamics required for successful virus-host cell fusion (e.g., Chernomordik et al., J Cell Biol, 175:201-7 (2006); Martin and Ruysschaert, Biochim Biophys Acta, 1240:95-100 (1995); Langosch et al., J Biol Chem, 276:32016-21 (2001); Langosch et al., Cell Mol Life Sci, 64:850-64 (2007)). For example, the phospholipid analog lysophosphotidylcholine (LPC) is designed to prevent the entry of certain enveloped viruses, such as influenza, HIV-1 (Class I fusion) and TBEV (Class II fusion), into host cell by stabilizing the positive spontaneous curvature of viral membranes and thereby preventing conformational changes needed for viral-host cell fusion (Chernomordik et al., J Cell Biol, 175:201-7 (2006); Chernomordik and Kozlov, Annu Rev Biochem, 72:175-207 (2003); Martin and Ruysschaert, Biochim Biophys Acta, 1240:95-100 (1995); Razinkov et al., J Gen Physiol, 112:409-22 (1998); Shangguan et al., Biochemistry, 35:4956-65 (1996); Gunther-Ausborn et al., J Biol Chem, 270:29279-85 (1995)). However, LPC's viability as a drug candidate is questionable since it exerts its effect in a highly reversible manner, requires high molar concentrations (10% or higher total lipid content), and can be effectively recycled and metabolized by cells.

n-docosanol, a 22-carbon saturated alcohol, is also designed to inhibit host cell entry of a variety of enveloped viruses (Katz et al., Proc Natl Acad Sci USA, 88:10825-9 (1991)). However, n-docosanol appears to inhibit virus-cell fusion by perturbing the properties of the target cell rather than the virus (Katz et al., Ann N Y Acad Sci, 724:472-88 (1994); Marcelletti et al., AIDS Res Hum Retroviruses, 12:71-4 (1996); Pope et al., Antiviral Res, 40:85-94 (1998)), as optimal inhibition is observed when cells, but not virus, are pre-incubated for several hours with n-docosanol. In addition, poor solubility and a millimolar IC₅₀ has limited n-docosanol to use as a 10% v/v topical microbicide (Abreva™) for the treatment of cold sores (Katz et al., Ann N Y Acad Sci, 724:472-88 (1994); Marcelletti et al., AIDS Res Hum Retroviruses, 12:71-4 (1996); Pope et al., Antiviral Res, 40:85-94 (1998)).

Recently, amphipathic peptides derived from the NS5A protein of Hepatitis C have been identified as having antiviral activity related to their ability to disrupt the membrane integrity of enveloped viruses (Bobardt et al., Proc Natl Acad Sci USA, 105:5525-30 (2008); Cheng et al., Proc Natl Acad Sci USA, 105:3088-93 (2008)). Although the NS5A-derived peptides were not active against all enveloped viruses, their broad range of activity validates viral membranes as a therapeutic target for broad spectrum antiviral therapeutics.

Thus, there is a need in the art for broad-spectrum antiviral drug capable of disrupting viral lipid membranes and thereby preventing viral infections.

### BRIEF DESCRIPTION OF DRAWINGS

FIG. 1. a) Pseudotyped VSV (pVSV) with the indicated envelope was pretreated with LJ001 or 0.1% DMSO (vehicle) for 10′ at 25° C., and then used to infect Vero cells for 1 h at 37° C. (±s.e.m., normalized DMSO at 100% between experiments). b) VSV-Indiana at an M.O.I. of 3 was treated as in a) and infection was quantified from supernatant samples using a standard plaque assay (±s.e.m.). NiV at an M.O.I. of 3 was treated as in a) with 10 μM LJ001 and TCID₅₀ measurements were taken and infectious innocula was replaced with growth media containing LJ001 at the indicated time points (representative experiment).

FIG. 2. a) In vitro VSV mRNA was synthesized using 20 μg of purified VSV-Indiana. mRNA was treated with 10 μM LJ001 or 0.1% DMSO, purified, and measured by radiodetection within an agarose-urea gel, as described in Li et al., J Virol, 79:13373-84 (2005). b) Total in vitro VSV mRNA, prepared as described in a), was treated with tobacco acid pryophosphatase (TAP) and subjected to thin layer chromatography to assay for cap methylation; SAH=S-adenosylhomocysteine.

FIG. 3. a) Vero cells were fully passaged (repeatedly rinsed, trypsinized, and split daily) in 10 μM of fresh LJ001 for 4 days and then visualized at 25× magnification under brightfield (representative images). b) Vero cells were treated with varying concentrations of LJ001 for 1 h at 37° C. and cells were assayed for lactate dehydrogenase (LDH) and adenylate kinase (AK) release (±s.d.). c) Vero cells were plated at ˜10% confluency and exposed to LJ001. Uptake of Alamar Blue substrate as cells proliferated (2 days) was measured by colorimetric measurements per manufacturer's instructions (±s.d.).

FIG. 4. a) 100 pfu of Ebola-Zaire (Cat. A, Filovirus) was treated with LJ001 or DMSO for 20′ at 25° C. and then used to infect VeroE6 cells for 1 h at 37° C. Plaques were counted at 10 d.p.i. (average of triplicates±s.d.). b) 5.0 ng of R5-HIV-1 (YU2) was treated with LJ001 or DMSO for 20′ at 25° C. and then used to infect 293-inducible cell lines, induced to express high levels of CD4/CCR5 at 37° C. At 8 h.p.i., cells were harvested and assayed for early reverse transcription products via quantitative real-time PCR (average of duplicates±s.d.). c) 80 pfu of La Crosse virus (Cat. B, Bunyavirus) was treated with LJ001 or DMSO for 20′ at 25° C. and then used to infect BHK-S cells for 1 h at 37° C. Plaques were counted at 4 d.p.i. (average of triplicates±s.d.). d) 120 pfu of Junín virus (Cat. A, Arenavirus) was treated with LJ001 or DMSO for 20′ at 25° C. and then used to infect VeroE6 cells for 1 h at 37° C. Plaques were counted at 5 d.p.i. (average of triplicates±s.d.). e) 50 ng of HIV-1 (pNL-GFPΔenv) psuedotyped with VSV-G was treated with 10 μM LJ001 or DMSO for 10′ at 25° C. and then used to infect 293T and Vero cells for 1 h at 37° C. At 5 d.p.i., cells were visualized under a fluorescent microscope (bottom panel) and then harvested and assayed for GFP positive cells via flow cytometry (average of triplicates±s.d.). Cellular entry of the lentiviral genome was inhibited regardless of the envelope. f) A 1:25 titration (resulting in ˜90% infection) of unconcentrated supernatant produced from Ad5-GFP infected cells was treated with 10 μM LJ001 or 0.1% DMSO for 10′ at 25° C. and then used to infect 293T and Vero cells for 1 h at 37° C. At 18 h.p.i., cells were visualized under a fluorescent microscope and then harvested and assayed for GFP positive cells via flow cytometry (average of triplicates±s.d.). No inhibition was seen with LJ001. Similar results were obtained for a recombinant Coxsackie-B-GFP virus. g) JFH1 strain of Hepatitis C Virus was pre-treated with 1 μM or 10 μM (not shown) LJ001 or LJ025 for 10′ at 25° C. and then used to infect Huh-7.5.1 cells. upper left (entry)) After a 72 h infection, cells were scored for HCV NS5A protein by immunofluorescence as a measure of virus replication. upper right (secondary infection)) The supernatants (secreted virus) from the entry experiment were used to infect naïve Huh-7.5.1 cells. After a 72 h infection, the cells were scored for HCV NS5A protein by immunofluorescence as a measure of virus replication. h) HPIV3 (MOI=0.1) was pre-treated with the indicated concentrations of LJ001 or DMSO for 20′ on ice and then used to infect CV-1 cells. After 90′ at 37° C.: lower left (entry)) medium containing virus and compounds was replaced with an overlay of agar-media and incubated overnight at 37° C., or lower right (secondary infection)) medium containing virus and compounds was replaced with regular media containing compounds at the same concentrations followed by overnight incubation at 37° C. At 12 and 24 hours post-infection, 10 μl and 100 μl from each condition was used for plaque assays as described above. i) 50 pfu/well of WNV was pre-treated with the indicated concentration of LJ001 for 20′ at 25° C. and then used to infect Vero-E6 cells for 1 h at 37° C. Virus innoculum was removed and cells were washed with PBS and overlain with tragacanth (average of quadruplicates±s.d.). j) 50 pfu/well of Reovirus (T3D) was pre-treated with LJ001, LJ025, or DMSO for 10′ at 25° C. and then used to infect MDCK cells for 1 h at 37° C. Virus innoculum was removed and cells were overlain with 1% bacto-agar in DMEM. After 7 days, cells were stained with 0.03% neutral red and visually assayed for plaque formation (average of duplicates±s.d.). k) A 1:500 dilution of NDV-GFP replication competent virus harvested from the allantoic fluid of embryonated chicken eggs (100 pfu/egg) was pretreated with the indicated concentration of LJ-compound and then used to infect Vero cells for 1 h at 37° C. The innoculum was then replaced with regular growth media and, after 18 h, cells were harvested, fixed, and assayed for GFP expression via flow cytometry (average of duplicates±s.d.). l) 50 pfu of Rift Valley Fever MP-12 virus (Cat. B, Bunyavirus) was treated with LJ001 for 20′ at 25° C. and then used to infect BHK-S cells for 1 h at 37° C. Plaques were counted 4 d.p.i. (average of triplicates±s.d.).

FIG. 5. a) Viruses were treated with LJ001 at 10 μM for 10′ at 25° C., washed with PBS, and repurified by ultracentrifugation through a sucrose cushion. Repurified viruses were used to infect cells as previously described (±s.d.). b) Viruses were treated with LJ001 at 10 μM for 10′ at 25° C., washed with 6 ml PBS, repurified by ultracentrifugation through a sucrose cushion, washed again for 4 h in 6 ml PBS, and then re-purified again. Repurified viruses were used to infect cells as previously described (±s.d.). c) Vero cells were treated with 1 μM or 10 μM compound for 10′, 30′, or 120′ at 37° C. in PBS (+10% FBS) and either left alone (no wash) or washed 3 times (3× wash), followed by infection with pVSV (individual data sets normalized to corresponding vehicle control or negative compound, ±s.d.). d) pVSV was used to infect Vero cells as previously described. 10 μM LJ001 was added at the indicated time, with relation to infection endpoint (±s.d.).

FIG. 6. a) 18 BALB/cAnNCrl female mice at 6-8 weeks of age were innoculated via intraperitoneal (IP) and oral gavage (OG) routes daily for 7 days with 50 μl of 100% DMSO or LJ001 (in 100% DMSO) at 20 mg/kg (low) or 50 mg/kg (high) doses (n=3 per group). Daily averaged weights of the animals in each group are shown (±s.d). b) On day 8, all animals were euthanized via CO₂ asphyxiation, terminal blood samples were collected via cardiac puncture, and complete CBC with differential and chemistry panel analyses were conducted. Results are shown as averages of 3 individual animals. c) Quantitative graph of cholesterol levels measured in b) (n=3).

FIG. 7. a) Liposomes were titered into a solution containing 10 μM LJ001 (excitation: 450 nm; emission: 510 nm) and fluorescence was monitored at the indicated wavelengths using a PTI QM4 fluorescence spectrophotometer. Fluorescence did not increase when titrating in identical concentrations of hydrophilic silica beads of a similar size (see FIG. 8a). Representative raw data are shown. Solid line=no liposomes; Dashed and Dotted lines=increasing liposomal titrations. b) A quantification of individual peaks at 510 nm (solid vertical line from FIG. 7a) as increasing concentrations of liposomes are titered into solution. Triton-X (0.1% final) was added at the end of the assay to show that the increasing fluorescence depended on intact liposomes. The data was corrected for scattering caused by the addition of liposomes by repeating the experiments in the absence of LJ001 and subtracting the liposome induced scattering signal (±s.e.m.) (see FIG. 8b). c) 25,000 Vero cells were stained with increasing concentrations of LJ001 (see flanking bar graph showing MFI values) for 30′ at 37° C. in normal growth media, then harvested by trypsinization or scraping and analyzed for mean fluorescence intensity (MFI) by flow cytometry (LJ001 peak ex: 460 nm em: 510 nm, laser: 488 nm, detector: 509 nm). Single representative experiment. d) Vero cells were infected with pVSV as previously described while being simultaneously subjected to 10 μM LJ001 and liposomes (±s.d.). e) Vero cells were infected with pVSV treated with 10 μM LJ001 for 10′ at 25° C., and then subjected to varying concentrations of liposomes (±s.d.). f) Purified pVSV was treated with 300 μM LJ001, incubated at 25° C. for 10′, and then stained with 2% phosphotungstic acid (PTA) and visualized by EM. Insets: 75,000× magnification, sizing bars=100 nm (representative images, arrows=disrupted particles, arrowheads=selected representative intact particles). g) Liposomes (0.15 mg/mL final concentration) were added to 10 μM R18 in PBS after 100 seconds. R18 was allowed to integrate into the membranes for 50′ and then 5 individual titrations of compound were added at 10 μM increments (‘+’). Each titration was allowed to equilibrate for 5′ prior to addition of the next titration. Signal prior to first titration was normalized to 1.0. To induce maximum particle disruption and obtain the maximum signal, 0.1% Triton-X (‘Tx’) was added at the end of the experiment. Data were collected using a PTI QM4 fluorescence spectrophotometer at 25° C. (with constant stirring) using a 4 nm excitation/emission bandpass at 560 nm excitation and 590 nm emission (representative experiment).

FIG. 8. a) 200 nm hydrophilic silica beads were assayed for fluorescence intensity binding in the presence of 10 μM LJ001 as described in relation to FIG. 7. b) Quantification of individual peaks at 510 nm (dotted line) with background scattering (solid vertical line) as increasing concentrations of liposomes are titered into solution in the presence of 10 μM LJ001. Triton-X (0.1% final conc.) was added at the end of the assay to show that increasing fluorescence depended on intact liposomes. c) A saturating amount of 200 nm (diameter) sized liposomes (8 mg/ml) was incubated with 10 μM LJ001 for 10′ at 25° C. Virus was added and the mixture was incubated for the indicated amount of time and then used to infect Vero cells. LJ001 was unable to exit the liposomes and inactivate nearby viral particles under the conditions examined (data normalized to corresponding DMSO timepoint; experiments conducted as in FIG. 7d-e, ±s.d.). d) Differentially sized liposomes of the same composition as those used in FIG. 7d-e were purified via size exclusion. Vero cells were infected with pVSV, as previously described, after being simultaneously pre-treated with 10 μM LJ001 and the indicated size and concentration of liposomes (±s.d., single representative experiment). e) Fluorescence intensity signal (510 nm) after titration of liposomes of the indicated size into 10 μM LJ001. Background scattering due to the presence of liposomes alone was subtracted as described in 7b. f) NDV was treated with LJ001 (left panel) or LJ025 (right panel) and visualized at 60,000× magnification (sizing bars represent 40 nm), 2% PTA.

FIG. 9. a) Pseudotyped VSV (pVSV+NiV−F/G) was pretreated with the indicated concentration of LJ001 or LJ033 for 10′ at 25° C., and then used to infect Vero cells for 1 h at 37° C. (±s.d., representative experiment). b) Fluorescence intensity signal after titration of liposomes into 10 μM LJ025 (LJ025 peak ex: 410 nm em:460 nm, laser: 405 nm, detector: 455 nm) was performed as described in the main text. Background scattering due to the presence of liposomes alone was subtracted as described in 7b. (±s.d.) c) 25,000 Vero cells were stained with increasing concentrations of LJ025 (see flanking bar graph showing MFI values) for 30′ at 37° C. in normal growth media, then harvested by trypsinization or scraping and analyzed for mean fluorescence intensity (MFI) by flow cytometry (ex: 401, em: 450). (Single representative experiment)

FIG. 10. a) RVFV MP-12 was treated with 10 μM LJ001 or 2.5% DMSO for 20′ at 25° C. and repurified across a continuous iodixanol (OptiPrep™, Sigma) density gradient, and envelope (GC/GN) and nucleocapsid (N) proteins were detected by immunoblotting. b) Fractions from 5a were used to conduct a plaque assay measuring infectivity (white bars=DMSO, solid bars=LJ001). LJ001 treated viruses exhibited at least a 5-log reduction in infectivity (representative experiment). c) CHO cells constitutively expressing ephrinB2 were incubated with NiV-pseudotyped pVSV in the presence of 0.1% DMSO, 10 μM LJ001, or 40 nM soluble ephrinB2-Fc (EFN-B2) at 4° C. for 2 h. Cells were washed and fixed in 0.5% PFA, and bound viruses were detected with anti-NiV-F at 4° C. for 45′. After several washes, the cells were incubated with goat-anti-rabbit conjugated to Alexa567 nm for 30′ at 4° C. The cells were then fixed in 2% PFA and quantified by flow cytometry (representative experiment). d) Graphical representation of the raw histogram data sets from 5c.

FIG. 11. a) NiV VLPs were produced by transfecting NiV-env proteins and a β-lactamase-matrix fusion protein into 293T cells. VLPs in supernatant were collected and concentrated via ultracentrifugation. Target cells were loaded with a cell permeant substrate, CCF2-AM, that contains 2 fluorophores linked by a β-lactam ring. Introduction of β-lactamase (fused to NiV-matrix), upon entry of the VLPs into the target cells, results in cleavage of the β-lactam ring within the CCF2-AM substrate, disrupting FRET between the fluorophores and resulting in a green to blue shift within infected cells. 10 μM LJ001- and DMSO-treated VLPs were used to infect Vero cells pre-loaded with CCF2-AM substrate, and the cells were assayed for infection via flow cytometry. Data is shown as normalized ratios of Blue:Green cells (±s.e.m.). b) Vero cells were transfected with NiV-F and G constructs, incubated overnight in media with 10 μM LJ001 or 0.1% DMSO, DAPI stained, and assayed visually for nuclei in syncytia by counting and averaging five 10× fields (±s.d.).

FIG. 12. Aliquots of virus equivalent to 100 times the LD₅₀ of RVFV-ZH501 or mouse-adapted Ebola-Zaire (maZEBOV) were treated ex vivo with 20 μM LJ001, 20 μM LJ025, or 2.5% DMSO for 20′ at 25° C., and then used to infect mice (RVFV, n=5; maZEBOV, n=5) via intraperitoneal injection. Representative data of two separate experiments are shown.

FIG. 13. HIV-1 (JRCSF) was passaged in the continuous presence of 6 μM LJ001 or LJ025 on GHOST-R5 cells. Viral replication cultures were split 1:4 twice weekly, and replication was monitored by the LTR-GFP reporter as well as p24 antigen level in the supernatant. After 8 consecutive passages (˜4 weeks), LJ001-passaged virus or LJ025-passaged virus were adjusted to 25 ng equivalent of p24 and tested for sensitivity to LJ001 inhibition on fresh Ghost-R5 cells. Cells were assayed for GFP production via flow cytometry (average of duplicates, ±s.d.).

FIG. 14. a) Post-challenge efficacy. 1000 pfu of ma-ZEBOV was used to infect female BALB/c mice (n=10) via intraperitoneal injection in a 0.2 ml volume. Immediately after challenge, the animals were dosed IP with 50 mg/kg of LJ001 in 100% DMSO at a dose volume of 50 μl. Mice were re-dosed every 24 h for 7d. b) Pharmacokinetics. Male Sprague-Dawley rats (n=3) were implanted with jugular vein catheters (JVC) and dosed singly IP with LJ001 at 20 mg/kg or 50 mg/kg. 300 μl blood samples were taken by JVC at the indicated time intervals and placed in K2-EDTA tubes. LJ001 was detected via LC/APCI-MSMS and quantified using penta-deuterated LJ001 as an internal standard. Surprisingly, the 20 mg/kg group gave a higher peak serum concentration than the 50 mg/kg group.

FIG. 15. Structures of compounds in the LJ series of arylmethylidene rhodanine derivatives.

### DETAILED DESCRIPTION

Provided herein are a series of arylmethylidene rhodanine derivatives having broad-spectrum antiviral activity. Without being limited to a particular theory, it is believed that the compounds are capable of binding and/or inserting into the outer membranes of enveloped viruses and disrupting the membrane structure and/or dynamics in a manner that inhibits viral attachment, fusion, and/or entry into host cells. Moreover, the membrane disrupting activity of the compounds is preferably selective for viral membranes relative to non-viral biological membranes, including but not limited to, cell plasma membranes, organelle membranes, biological barriers, and other non-viral lipid bilayers. Thus, compounds provided herein are substantially non-toxic and have a wide therapeutic index for antiviral activity in vivo without significant toxicity or side effects. Because membranes are common to all enveloped viruses, compounds provided herein have broad ranging antiviral activity against enveloped viruses, including but not limited to, filoviruses, poxviruses, arenaviruses, bunyaviruses, paramyxoviruses, flaviviruses, influenza A, and HIV-1.

In some aspects, antiviral compounds of formula I, and pharmaceutically acceptable salts, prodrugs, and derivatives thereof, are provided herein

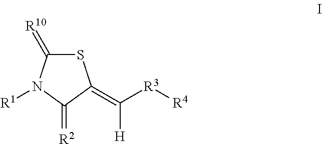

wherein

R¹ is H; C₁-C₆ alkyl, C₂-C₆ alkenyl, C₂-C₆ alkynyl, C₁-C₆ alkoxy, C₃-C₆ aryl, C₃-C₆ heteroaryl, C₃-C₆ cycloalkyl, or C₃-C₆ heterocycloalkyl, each optionally substituted with halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷; halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷;

R² is O or S;

R³ is C₅ heteroaryl;

R⁴ is C₅-C₆ aryl or C₅-C₆ heteroaryl, each optionally substituted with C₁-C₆ alkyl, C₂-C₆ alkenyl, C₂-C₆ alkynyl, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷;

R⁵, R⁶, and R⁷ are independently H, C₁-C₆ alkyl, C₂-C₆ alkenyl, C₂-C₆ alkynyl, C₁-C₆ alkoxy, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷; and

R¹⁰ is O or S when R² is S, and R¹⁰ is S when R² is O.

In some aspects, R² is O.

In additional aspects, R² is S.

In some preferred aspects, R³ is pyrrolyl, thienyl, furanyl, pyrazolyl, imidazolyl, thiazolyl, isothiazolyl, oxazolyl, or isoxazolyl.

In further preferred aspects, R³ is pyrrolyl, thienyl, or furanyl.

In some preferred aspects, R⁴ is phenyl, pyranyl, thiopyranyl, pyridyl, pyrimidinyl, pyrazinyl, or pyridazinyl.

In further preferred aspects, R⁴ is phenyl.

Also provided herein are antiviral compounds of formula II, and pharmaceutically acceptable salts, prodrugs, and derivatives thereof

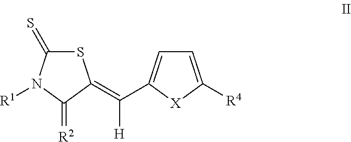

wherein X is O, N, or S.

In some preferred aspects, X is O.

In further preferred aspects, R² is O.

In yet further preferred aspects, R² is S.

Also provided herein are compounds of formula III, and pharmaceutically acceptable salts, prodrugs, and derivatives thereof.

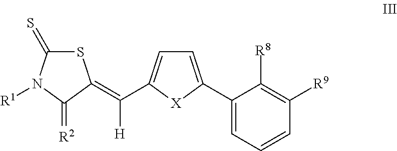

wherein X is O, N, or S; and

R⁸ and R⁹ are independently H, C₁-C₆ alkyl, C₂-C₆ alkenyl, C₂-C₆ alkynyl, C₁-C₆ alkoxy, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷.

In some preferred aspects, R⁸ and R⁹ are independently H, C₁-C₆ alkyl, —OR⁵, —SR⁵, or —NO₂.

In some preferred aspects, R² is O.

In further preferred aspects, R² is S.

In some aspects, the antiviral compound of formula I, II, and/or III is: (Z)-3-allyl-5-(5-phenyl-2-furyl)methylene-4-oxothiazolidine-2-thione (LJ-001); (Z)-3-ethyl-5-(5-phenyl-2 furyl)methylene-4-oxothiazolidine-2-thione (LJ-002); (Z)-3-propyl-5-(5-phenyl-2-furyl)methylene-4-oxothiazolidine-2-thione (LJ-003); (Z)-3-benzyl-5-(5-phenyl-2-furyl)methylene-4-oxothiazolidine-2-thione (LJ-004); (Z)-3-methyl-5-(5-phenyl-2-furyl)methylene-4-oxothiazolidine-2-thione (LJ-005); (Z)-3-Ethyl-5-[5-(3-chlorophenyl)-2-furyl]methylene-4-oxothiazolidine-2-thione (LJ-006); (Z)-3-Ethyl-5-[5-(3-fluorophenyl)-2-furyl]methylene-4-oxothiazolidine-2-thione (LJ-007); (Z)-3-Ethyl-5-[5-(2-fluorophenyl)-2-furyl]methylene-4-oxothiazolidine-2-thione (LJ-008); (Z)-3-Ethyl-5-[5-(2-chlorophenyl)-2-furyl]methylene-4-oxothiazolidine-2-thione (LJ-009); (Z)-3-Ethyl-5-[5-(2-methoxyphenyl)-2-furyl]methylene-4-oxothiazolidine-2-thione (LJ-010); (Z)-3-Ethyl-5-[5-(3-methoxyphenyl)-2-furyl]methylene-4-oxothiazolidine-2-thione (LJ-011); (Z)-3-Ethyl-5-[5-(2-trifluoromethylphenyl)-2-furyl]methylene-4-oxothiazolidine-2-thione (LJ-012); (Z)-3-ethyl-5-((5-(2-nitrophenyl)furan-2-yl)methylene)-2-thioxothiazolidin-4-one (LJ-015); (Z)-3-Ethyl-5-((5-(2-hydroxyphenyl)furan-2-yl)methylene)-2-thioxothiazolidin-4-one (LJ-016); (Z)-5-((5-(2-Aminophenyl)furan-2-yl)methylene)-3-ethyl-2-thioxothiazolidin-4-one (LJ-017); (Z)-3-ethyl-5-((5-phenylthiophen-2-yl)methylene)-2-thioxothiazolidin-4-one (LJ-018); (Z)-3-(4-Oxo-5-((5-phenylfuran-2-yl)methylene)-2-thioxothiazolidin-3-yl)propyl acetate (LJ-021); (Z)-3-(3-Hydroxypropyl)-5-((5-phenylfuran-2-yl)methylene)-2-thioxothiazolidin-4-one (LJ-022); (Z)-3-Ethyl-5-((2-phenyloxazol-5-yl)methylene)-2-thioxothiazolidin-4-one (LJ-023); (Z)-3-(4-Oxo-5-((5-phenylfuran-2-yl)methylene)-2-thioxothiazolidin-3-yl)propyl)-5-(2-oxohexahydro-1H-thieno[3,4-d]imidazol-4-yl)pentanoate (LJ-024); (Z)-3-(2-Propenyl)-5-((5-phenylfuran-2-yl)methylene-2-thioxothiazolidin-4-thione (LJ-027); (Z)-3-ethyl-5-((5-(2-nitrophenyl)furan-2-yl)methylene-2-thioxothiazolidin-4-thione (LJ-028); (Z)-5-(5-phenyl-2-furyl)methylene-4-oxothiazolidine-2-thione (LJ-031); (Z)-3-(2-propynyl)-5-(5-phenyl-2-furyl)methylene-4-oxothiazolidine-2-thione (LJ-032); (Z)—N-(3-(4-Oxo-5-((5-phenylfuran-2-yl)methylene)-2-thioxothiazolidin-3-yl)propyl)acetamide (LJ-034); (Z)-3-(3-Aminopropyl)-5-((5-phenylfuran-2-yl)methylene)-4-oxothiazolidin-2-thione (LJ-035); (Z)-tert-Butyl 3-(4-oxo-5-((5-phenylfuran-2-yl)methylene)-2-thioxothiazolidine-3-yl)propyl carbamate (LJ-036); or (Z)-3-(2,4-Dioxo-5-((5-phenylfuran-2-yl)methylene)thioxothiazolidin-3-yl) propyl-5-(2-oxohexahydro-1H-thieno[3,4-d]imidazol-4-yl)pentanoate (LJ-037).

In additional aspects, compounds of formula IV are provided herein along with pharmaceutically acceptable salts, prodrugs, and derivatives thereof

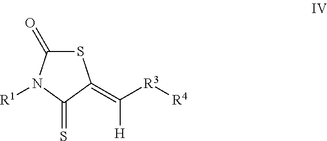

wherein

R¹ is H; C₁-C₆ alkyl, C₂-C₆ alkenyl, C₂-C₆ alkynyl, C₁-C₆ alkoxy, C₃-C₆ aryl, C₃-C₆ heteroaryl, C₃-C₆ cycloalkyl, or C₃-C₆ heterocycloalkyl, each optionally substituted with halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷; halo, —NO₂, —CF₃, —CN, —OR⁵, —SR⁵, —C(O)R⁵—NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷;

R³ is C₅ heteroaryl;

R⁴ is C₅-C₆ aryl or C₅-C₆ heteroaryl, each optionally substituted with C₁-C₆ alkyl, C₂-C₆ alkenyl, C₂-C₆ alkynyl, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷; and

R⁵, R⁶, and R⁷ are independently H, C₁-C₆ alkyl, C₂-C₆ alkenyl, C₂-C₆ alkynyl, C₁-C₆ alkoxy, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷.

In some preferred aspects, R³ is pyrrolyl, thienyl, furanyl, pyrazolyl, imidazolyl, thiazolyl, isothiazolyl, oxazolyl, or isoxazolyl.

In further preferred aspects, R³ is pyrrolyl, thienyl, or furanyl.

In some preferred aspects, R⁴ is phenyl, pyranyl, thiopyranyl, pyridyl, pyrimidinyl, pyrazinyl, or pyridazinyl.

In further preferred aspects, R⁴ is phenyl.

In further aspects, compounds of formula V are provided herein along with pharmaceutically acceptable salts, prodrugs, and derivatives thereof

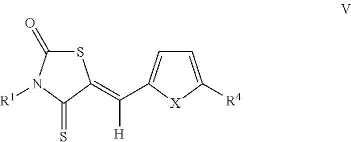

wherein X is O, N, or S.

In some preferred aspects, X is O.

Also provided herein are compounds of formula VI, and pharmaceutically acceptable salts, prodrugs, and derivatives thereof

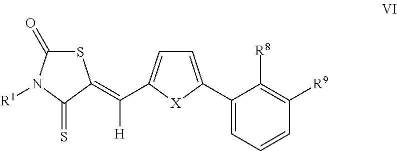

wherein X is O, N, or S; and

R⁸ and R⁹ are independently H, C₁-C₆ alkyl, C₂-C₆ alkenyl, C₂-C₆ alkynyl, C₁-C₆ alkoxy, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷.

In some preferred aspects, R⁸ and R⁹ are independently H, C₁-C₆ alkyl, —OR⁵, —SR⁵, or —NO₂.

In some aspects, the antiviral compound of formula IV, V, and/or VI is: (Z)-3-allyl-5-(5-phenyl-2-furyl)methylene-2-oxothiazolidine-4-thione (LJ-029); (Z)-3-ethyl-5-((5-(2-nitrophenyl)furan-2-yl)methylene)-2-oxothiazolidin-4-thione (LJ-030); (Z)-3-ethyl-5-(5-phenyl-2 furyl)methylene-2-oxothiazolidine-4-thione; (Z)-3-propyl-5-(5-phenyl-2-furyl)methylene-2-oxothiazolidine-4-thione; (Z)-3-benzyl-5-(5-phenyl-2-furyl)methylene-2-oxothiazolidine-4-thione; (Z)-3-methyl-5-(5-phenyl-2-furyl)methylene-2-oxothiazolidine-4-thione; (Z)-3-Ethyl-5-[5-(3-chlorophenyl)-2-furyl]methylene-2-oxothiazolidine-4-thione; (Z)-3-Ethyl-5-[5-(3-fluorophenyl)-2-furyl]methylene-2-oxothiazolidine-4-thione; (Z)-3-Ethyl-5-[5-(2-fluorophenyl)-2-furyl]methylene-2-oxothiazolidine-4-thione; (Z)-3-Ethyl-5-[5-(2-chlorophenyl)-2-furyl]methylene-2-oxothiazolidine-4-thione; (Z)-3-Ethyl-5-[5-(2-methoxyphenyl)-2-furyl]methylene-2-oxothiazolidine-4-thione; (Z)-3-Ethyl-5-[5-(3-methoxyphenyl)-2-furyl]methylene-2-oxothiazolidine-4-thione; (Z)-3-Ethyl-5-[5-(2-trifluoromethylphenyl)-2-furyl]methylene-2-oxothiazolidine-4-thione; (Z)-3-Ethyl-5-((5-(2-hydroxyphenyl)furan-2-yl)methylene)-2-oxothiazolidin-4-thione; (Z)-5-((5-(2-Aminophenyl)furan-2-yl)methylene)-3-ethyl-2-oxothiazolidin-4-thione; (Z)-3-ethyl-5-((5-phenylthiophen-2-yl)methylene)-2-oxothiazolidin-4-thione; (Z)-3-(4-Thio-5-((5-phenylfuran-2-yl)methylene)-2-oxothiazolidin-3-yl)propyl acetate; (Z)-3-(3-Hydroxypropyl)-5-((5-phenylfuran-2-yl)methylene)-2-oxothiazolidin-4-thione; (Z)-3-Ethyl-5-((2-phenyloxazol-5-yl)methylene)-2-oxothiazolidin-4-thione; (Z)-3-(2-Oxo-5-((5-phenylfuran-2-yl)methylene)-4-thioxothiazolidin-3-yl)propyl)-5-(2-oxohexahydro-1H-thieno[3,4-d]imidazol-4-yl)pentanoate; (Z)-5-(5-phenyl-2-furyl)methylene-2-oxothiazolidine-4-thione; (Z)-3-(2-propynyl)-5-(5-phenyl-2-furyl)methylene-2-oxothiazolidine-4-thione; (Z)—N-(3-(2-Oxo-5-((5-phenylfuran-2-yl)methylene)-4-thioxothiazolidin-3-yl)propyl)acetamide; (Z)-3-(3-Aminopropyl)-5-((5-phenylfuran-2-yl)methylene)-2-oxothiazolidin-4-thione; or (Z)-tert-Butyl 3-(2-oxo-5-((5-phenylfuran-2-yl)methylene)-4-thioxothiazolidine-3-yl)propyl carbamate.

Chemical moieties referred to as univalent chemical moieties (e.g., alkyl, aryl, etc.) also encompass structurally permissible multivalent moieties, as understood by those skilled in the art. For example, while an “alkyl” moiety generally refers to a monovalent radical (e.g., CH₃CH₂—), in appropriate circumstances an “alkyl” moiety can also refer to a divalent radical (e.g., —CH₂CH₂—, which is equivalent to an “alkylene” group). Similarly, under circumstances where a divalent moiety is required, those skilled in the art will understand that the term “aryl” refers to the corresponding divalent arylene group.

All atoms are understood to have their normal number of valences for bond formation (e.g., 4 for carbon, 3 for N, 2 for O, and 2, 4, or 6 for S, depending on the atom's oxidation state). On occasion a moiety may be defined, for example, as (A)ₐB, wherein a is 0 or 1. In such instances, when a is 0 the moiety is B and when a is 1 the moiety is AB.

Where a substituent can vary in the number of atoms or groups of the same kind (e.g., alkyl groups can be C₁, C₂, C₃, etc.), the number of repeated atoms or groups may be represented by a range (e.g., C₁-C₆ alkyl) which includes each and every number in the range and any and all sub ranges. For example, C₁-C₃ alkyl includes C₁, C₂, C₃, C₁₋₂, C₁₋₃, and C₂₋₃ alkyl.

The terms “alkyl,” “alkenyl,” and “alkynyl,” refer to straight and branched chain aliphatic groups having from 1 to 30 carbon atoms, or preferably from 1 to 15 carbon atoms, or more preferably from 1 to 6 carbon atoms, each optionally substituted with one, two or three substituents depending on valency. Examples of such groups include, without limitation, methyl, ethyl, propyl, isopropyl, butyl, tert-butyl, isobutyl, pentyl, hexyl, vinyl, allyl, isobutenyl, ethynyl, and propynyl.

The term “cycloalkyl” includes saturated and partially unsaturated cyclic hydrocarbon groups having from 3 to 12, or preferably from 3 to 8, or more preferably from 3 to 6 carbon atoms, each optionally substituted with one or more substituents. Examples of cycloalkyl groups include, without limitation, cyclopropyl, cyclobutyl, cyclopentyl, cyclopentenyl, cyclohexyl, cyclohexenyl, cycloheptyl, and cyclooctyl.

An “aryl” group is an optionally substituted C₆-C₁₄ moiety comprising one to three aromatic rings. In some aspects, the aryl group is a C₆-C₁₀ aryl group, or more preferably a C₅-C₆ aryl group. Examples of aryl groups include, without limitation, phenyl, naphthyl, anthracenyl, and fluorenyl.

A “heterocyclic” or “heterocyclyl” substituent is a non-aromatic mono-, bi-, or tricyclic structure having from about 3 to about 14 atoms, including one or more heteroatoms selected from N, O, and S. One ring of a bicyclic heterocycle or two rings of a tricyclic heterocycle may be aromatic (e.g., as in indan and 9,10-dihydro anthracene). Heterocyclic groups can be optionally substituted on one or more carbon, oxygen, nitrogen and/or sulfur atoms. Examples of heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino.

A “heteroaryl” group is an aromatic ring or ring system having about 5 to 14 ring atoms, or more preferably 5, 6, 9, or 10 ring atoms, including one or more heteroatoms selected from the group consisting of N, O, and S; and 6, 10, or 14 μl electrons shared in a cyclic array. Examples of heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

A “substituted” moiety is a moiety in which one or more hydrogen atoms have been independently replaced with another chemical substituent. As a non limiting example, substituted phenyl groups include 2-fluorophenyl, 3,4-dichlorophenyl, 3-chloro-4-fluorophenyl, and 2-fluoro-3-propylphenyl. In some instances, a methylene group (—CH₂—) is substituted with oxygen to form a carbonyl group (—CO).

An “optionally substituted” group can be substituted with from one to four, or preferably from one to three, or more preferably one or two non-hydrogen substituents. Examples of suitable substituents include, without limitation, alkyl, alkenyl, alkynyl, cycloalkyl, heterocycloalkyl, aryl, heteroaryl, aroyl, halo, hydroxy, oxo, nitro, alkoxy, amino, imino, azido, mercapto, acyl, carbamoyl, carboxy, carboxamido, amidino, guanidino, sulfonyl, sulfinyl, sulfonamido, formyl, cyano, and ureido groups.

The term “halogen” or “halo” refers to chlorine, bromine, fluorine, or iodine.

The term “acyl” refers to an alkylcarbonyl or arylcarbonyl substituent.

The term “acylamino” refers to an amide substituent attached to the structure at the nitrogen atom. Acylamino groups may be optionally substituted.

The term “carbamoyl” refers to an amide substituent attached to the structure at the carbonyl carbon atom. Carbamoyl groups may be optionally substituted.

The term “sulfonamido” refers to a sulfonamide substituent attached to the structure by either the sulfur or the nitrogen atom.

Unless otherwise specified, compounds provided herein include all of their various stereochemical forms, including but not limited to, enantiomers, diastereomers, rotamers, and the like. Also, moieties disclosed herein which exist in multiple tautomeric forms include all such forms encompassed by a given tautomeric structure.

Particular geometric isomers (e.g., E or Z isomers) disclosed herein include the E or Z isomer substantially free from the other isomer as well as mixtures of E and Z isomers in varying ratios. For example, in some preferred aspects, compounds provided herein comprise the (Z)-isomer substantially free from the (E)-isomer.

Certain E and Z geometric isomers can be interconverted by photolysis, photo irradiation or exposure to free radicals or certain solvents (see e.g., Ishida et al., Tetrahedron Lett 30:959 (1989)). For example, exposure of some (E) compounds to DMSO facilitates their conversion to the Z form.

Compounds provided herein can form useful salts with inorganic and organic acids, such as hydrochloric, sulfuric, acetic, lactic, or the like, and with inorganic or organic bases such as sodium or potassium hydroxide, piperidine, morpholine, ammonium hydroxide, or the like. Pharmaceutically acceptable salts of compounds provided herein can be prepared using procedures familiar to those skilled in the art.

Also provided herein are pharmaceutical compositions comprising an antiviral compound described herein and at least one pharmaceutically acceptable excipient.

The term “pharmaceutically acceptable excipient” includes any and all salts, buffering agents, preservatives, solvents, diluents, carriers, liquid vehicles, dispersion or suspension aids, surface active agents, isotonic agents, thickening or emulsifying agents, preservatives, solid binders, lubricants and the like which are compatible with the antiviral compounds provided herein and suitable for the particular dosage form desired. Various carriers, formulations, and techniques are described, e.g., in Remington's Pharmaceutical Sciences, E. W. Martin (Mack Publishing Co., Easton, Pa.).

Pharmaceutical compositions provided herein are formulated to be compatible with their intended route of administration. Exemplary routes of administration include, e.g., parenteral, intravenous, intramuscular, intradermal, subcutaneous, oral (e.g., inhalation), transdermal (topical), transmucosal, genital, vaginal, cervicovaginal and rectal administration.

Solutions or suspensions used for parenteral, intradermal, or subcutaneous application can include, e.g., a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

In some aspects, compounds and compositions provided herein are administered topically, e.g., by transmucosal or transdermal means. Suitable formulations for topical administration, including, e.g., vaginal or rectal administration, include solutions, suspensions, gels, lotions and creams as well as discrete units such as suppositories and microencapsulated suspensions. For transmucosal or transdermal administration, penetrants appropriate to the barrier to be permeated can be used. Such penetrants are generally known in the art, and include, for example, detergents, bile salts, and filsidic acid derivatives for transmucosal administration. Transmucosal administration can be accomplished through the use of nasal sprays, suppositories or transdermal formulations comprising active compounds formulated with ointments, salves, gels, creams, or the like.

In further aspects, compositions provided herein can be formulated as tablets, capsules or elixirs for oral administration or as sterile solutions or suspensions for injectable administration.

In some aspects, a compounds described herein is formulated as a sustained release composition which provides for slow, sustained release of the compound by a desired mode of administration. Such formulations can take the form of a sustained release gel, cream, suppository, capsule, or the like. In some aspects, active compounds are formulated within a system of carriers and excipients that protect the compound against rapid elimination from the body. Examples of such sustained release systems include: (a) erosional systems in which the active compound is contained within a matrix, and (b) diffusional systems in which the active component permeates at a controlled rate through a biocompatible polymer, such as ethylene vinyl acetate, polyanhydrides, polyglycolic acid, collagen, polyorthoesters, or polylactic acid. Methods for preparation of such formulations are known to those skilled in the art.

Pharmaceutical compositions provided herein can be utilized in conjunction with a delivery device, such as a condom or other contraceptive device, a metered dose inhaler, a transdermal patch, an implantable pump, sponge, or other reservoir, or the like.

In some aspects, pharmaceutical compositions provided herein may be delivered via an intranasal spray, by inhalation, and/or by an aerosol. Methods for delivering pharmaceutical compositions directly to the lungs and/or nasal mucosa via nasal and/or pulmonary aerosols are well-known in the pharmaceutical arts. In further aspects, pharmaceutical compositions provided herein may be delivered ocularly, e.g., via eyedrops.

In some preferred aspects, pharmaceutical compositions provided herein are delivered via a liposomal nanoparticle formulation. For example, in some aspects, the compounds can be formulated within liposomes comprising a lipid bilayer formulated to enhance solubility and/or permeability across viral membranes. In further aspects, a liposomal nanoparticle formulation provided herein can comprise liposomes having a size range which facilitates delivery of the active compounds to viral membranes. Liposomal formulations can be prepared according to methods known to those skilled in the art.

Pharmaceutical compositions suitable for injectable use include sterile aqueous solutions (for water soluble compounds) or dispersions and sterile powders for the extemporaneous preparation of sterile injectable solutions or dispersions.

For intravenous administration, suitable carriers include, e.g., physiological saline, bacteriostatic water, Cremophor EL™ (BASF, Parsippany, N.J.) or phosphate buffered saline (PBS). The compositions are preferably sterile and fluid to allow for easy syringability.

Oral compositions generally include an inert diluent or an edible carrier, and can be incorporated with excipients in the form of tablets, troches, capsules, or the like. Oral compositions can also be prepared using a fluid carrier for use as a mouthwash or rinse wherein the compound in the fluid carrier is applied orally and swished and expectorated or swallowed. Tablets, pills, capsules, troches and the like can further comprise one or more of the following: binding agents, such as microcrystalline cellulose, gum tragacanth or gelatin; adjuvants, such as starch or lactose, disintegrating agents, such as alginic acid, Primogel, or corn starch; lubricants, such as magnesium stearate or Sterotes; glidants, such as colloidal silicon dioxide; and sweetening or flavoring agents, such as sucrose, saccharin, peppermint, methyl salicylate, or orange flavoring.

Pharmaceutical compositions provided herein are preferably stable under the conditions of manufacture and storage and preserved against the contaminating action of microorganisms such as bacteria and fungi. The carrier can be a solvent or dispersion medium containing, for example, water, ethanol, polyol (e.g., glycerol, propylene glycol, liquid polyethylene glycol, or the like), and suitable mixtures thereof. The proper fluidity can be maintained, for example, by the use of a coating such as lecithin, by the maintenance of the required particle size in the case of a dispersion, and by the use of surfactants. Prevention of the action of microorganisms can be achieved by various antibacterial and antifungal agents, including, for example, parabens, chlorobutanol, phenol, ascorbic acid, thimerosal, and the like.

In some aspects, pharmaceutical compositions provided herein are formulated in dosage unit form (physically discrete units comprising a unitary, predetermined quantity of active compound calculated to produce the desired therapeutic effect in association with the required pharmaceutical carrier). The exact specifications of dosage unit forms will be dictated by the unique characteristics of the active compound, the particular therapeutic effect to be achieved, the preferred route of administration, and the like.

Toxicity and therapeutic efficacy antiviral compounds provided herein can be determined using standard pharmaceutical procedures in cell cultures or experimental animals. For example, established methods can be used to calculate LD₅₀ (the dose lethal to 50% of the population) and/or ED₅₀ (the dose therapeutically effective in 50% of the population) doses for the antiviral compounds. The dose ratio between toxic and therapeutic effects is the therapeutic index, which can be expressed as the ratio LD₅₀/ED₅₀. Compounds which exhibit large therapeutic indices are generally preferred, as are formulations and modes of administration which enhance the therapeutic index for a particular compound.

Data obtained from the cell culture assays and animal studies can be used in formulating a range of dosage for use in humans. In some aspects, dosages of antiviral compounds provided herein lie within a range of circulating concentrations which include the ED₅₀ with little or no toxicity. Dosages may vary within this range depending upon the dosage form, route of administration, and the like.

Therapeutically effective doses of compounds provided herein can be estimated initially from cell culture assays, e.g., based on the IC₅₀ (the concentration of a test compound which achieves a half-maximal inhibition of viral activity and/or infection). For example, the IC₅₀ observed in cell culture assays may be used to formulate a working dosage range for use in animal models in order to achieve a circulating plasma concentration range that includes the IC₅₀ with little or no toxicity. Conversion factors and calculation methods for converting animal dosages to human dose estimates are well known in the pharmaceutical arts. Concentrations of free compounds in plasma may be measured, for example, by high performance liquid chromatography.

As defined herein, a therapeutically effective amount of an antiviral compound provided herein (an effective dosage) can range from about 0.001 to 3000 mg/kg body weight, preferably from about 0.01 to 2500 mg/kg body weight, more preferably about 0.1 to 2000 mg/kg body weight, and even more preferably about 1 to 1000 mg/kg, 5 to 500 mg/kg, 10 to 100 mg/kg body weight. Skilled artisans will appreciate that a variety of factors may influence the dosage required to effectively treat a subject, including but not limited to the severity of the disease or condition being treated, history of previous treatments, general health and/or age of the subject, and the like. Accordingly, exact dosages for any particular subject will typically be determined empirically.

Pharmaceutical compositions provided herein may comprise a compound provided herein in an amount of at least 0.5% and generally not more than 90% by weight, based on the total weight of the composition, including excipients, if any. In some aspects, the proportion of antiviral agent varies between about 5-50% by weight of the composition.

Also provided herein are methods for treating or preventing infection by an enveloped virus, comprising administering an effective amount of a compound described herein to a subject in need of treatment.

As used herein, “treating” includes prevention, amelioration, alleviation, and/or elimination of a disease, disorder, or condition being treated or one or more symptoms of a disease, disorder, or condition being treated, as well as improvement in the overall well being of a patient, as measured by objective and/or subjective criteria.

In some aspects, the subject has been infected or is at risk of infection by an enveloped virus. A subject at risk of an enveloped virus infection can include any subject that has been exposed to or is likely to become exposed to an enveloped virus (e.g., via the skin or mucosal membranes). For example, subjects at risk can include medical providers, hospital staff and family members having contact with infected patients, as well as laboratory or quarantine facility workers having contact with samples, tissues, and like from infected patients.

In some aspects, the antiviral activity of a compound provided herein within a subject can be measured by assaying viral replication, viral infectivity, and/or viral load, and/or by measuring one or more secondary indicators of viral infection, such as indicators of inflammatory and/or immune responses.

Compounds and methods provided herein are useful for treating and preventing infections by any enveloped virus. “Enveloped” viruses are animal viruses having an outer membrane or ‘envelope’ comprised of a lipid bilayer with embedded viral proteins.

In some aspects, the enveloped virus is a type I Filoviridae virus which has a single-stranded, unsegmented (−) sense RNA genome and which causes severe hemorrhagic fever in humans and non-human primates. In some aspects, the Filoviridae virus is an Ebola virus, such as a Cote d'lvoire (CI), Sudan (S), Zaire (Z) or Reston (R) species of Ebola virus. In further aspects, the Filoviridae virus is a Marburg virus.

In some aspects, the virus is an Orthomyxoviridae virus, such as an influenza virus, Thogotovirus, Dhori virus, or infectious salmon anemia virus. For example, in some aspects, methods provided herein are used to treat or prevent infection of a human subject with an influenza type A virus, an influenza type B virus, or an influenza type C virus. In some aspects, the influenza type A virus is of subtype H1N1, H2N2, H3N2 or H5N1.

In some aspects, the virus is a Paramyxoviridae virus, such as human parainfluenza virus, human respiratory syncytial virus (RSV), Sendai virus, Newcastle disease virus, mumps virus, rubeola (measles) virus, Hendra virus, Nipah virus, avian pneumovirus, or canine distemper virus.

In some aspects, the virus is a Rhabdoviridae virus, such as rabies virus, vesicular stomatitis virus (VSV), Mokola virus, Duvenhage virus, European bat virus, salmon infectious hematopoietic necrosis virus, viral hemorrhagic septicaemia virus, spring viremia of carp virus, or snakehead rhabdovirus.

In some aspects, the virus is a Bornaviridae virus, such as Borna disease virus.

In some aspects, the virus is a Bunyaviridae virus, such as Bunyamwera virus, Hantaan virus, Crimean Congo virus, California encephalitis virus, Rift Valley fever virus, or sandfly fever virus.

In some aspects, the virus is an Arenaviridae virus, such as Old World Arenaviruses, Lassa fever virus, Ippy virus, Lymphocytic choriomeningitis virus (LCMV), Mobala virus, Mopeia virus, or a New World Arenavirus, such as Junin virus (Argentine hemorrhagic fever), Sabia (Brazilian hemorrhagic fever), Amapari virus, Flexal virus, Guanarito virus (Venezuela hemorrhagic fever), Machupo virus (Bolivian hemorrhagic fever), Latino virus, Boliveros virus, Parana virus, Pichinde virus, Pirital virus, Tacaribe virus, Tamiami virus, or Whitewater Arroyo virus. In some aspects, the Arenaviridae virus is Lymphocytic choriomeningitis virus, Lassa virus, Junin Virus, Machupo Virus, Sabia virus, or Guanarito virus.

In some aspects, the virus is an arbovirus. Arboviruses comprise a large group of more than 400 enveloped RNA viruses that are transmitted primarily by arthropod vectors (e.g., mosquitoes, sand-flies, fleas, ticks, lice, etc). In some aspects, the arbovirus is a Togaviridae virus, such as an Alphavirus (e.g. Venezuela equine encephalitis virus or Sindbis virus) or a Rubivirus (e.g. Rubella virus). For example, in some aspects, a compound provided herein is administered to a pregnant subject to treat or prevent congenital rubella syndrome (CRS) and symptoms related thereto, such as low birth weight, deafness, and abortion.

In some aspects, the arbovirus is a Flaviviridae virus, such as a Flavivirus, a Pestivirus, a Hepadvirus, yellow fever virus, dengue fever virus, or Japanese encaphilitis (JE) virus.

In some aspects, the virus is a Hepacivirus, such as a hepatitis C virus or a hepatitis C-like virus.

In some aspects, the virus is a Henipavirus, such as Hendra virus or Nipah virus.

In further aspects, the virus is a Bunyaviridae (−)-sense RNA virus, such as an Orthobunyavirus, a Hantavirus, a Phlebovirus, or a Nairovirus.

In some aspects, the virus is a Arenavirius virus, such as Lymphocytic choriomeningitis virus (LCMV), Lassa virus, Junin virus, Machupo virus, or Guanarito virus.

In some aspects, the virus is a Japanese encephalitis virus, such as Alfuy virus, Japanese encephalitis virus, Kokobera virus, Koutango virus, Kunjin virus, Murray Valley encephalitis virus, St. Louis encephalitis virus, Stratford virus, Usutu virus, or West Nile virus.

In some aspects, the virus is human immunodeficiency virus (HIV).

In some aspects, the virus is a herpesvirus, for example, HSV-1 or HSV-2.

Also provided herein are methods of treating a disease or condition associated with an enveloped virus infection. For example, in some aspects, methods are provided herein for treating Ebola Hemorrhagic Fever (EHF), Marburg hemorrhagic fever (MHF), Dengue fever, Dengue hemorrhagic fever (DHF), yellow fever, dengue fever, acute and chronic hepatitis C, Venezuelan hemorrhagic fever, Brazilian hemorrhagic fever, Bolivian hemorrhagic fever, lymphocytic choriomeningitis, Lassa fever, hantavirus pulmonary syndrome (HPS), meningitis, influenza, AIDS, and/or genital herpes.

In some aspects, a compound provided herein is administered in combination with an antiviral agent or an antiviral vaccine.

Compounds and compositions provided herein may be administered in any amount and via any route of administration effective for attenuating infectivity of an enveloped virus. Exemplary routes of administration include, but are not limited to, oral, intrathecal, intra-arterial, direct bronchial application, parenteral (e.g. intravenous), intramuscular, intranasal, sublingual, intratracheal, inhalation, ocular, vaginal, and rectal administration.

The term effective amount refers to the amount necessary or sufficient to realize a desired biologic effect. As understood by those skilled in the art, an effective amount for any particular application can vary depending on such factors as the disease or condition being treated, the particular inhibitor being administered, the size of the subject, the severity of the disease or condition being treated, and/or other factors.

In some aspects, an effective amount of a compound provided herein is an amount that, when administered via a preferred mode of administration, is effective to treat or prevent viral infection in the subject without causing substantial toxicity or adverse side-effects. In further aspects, an effective amount of an antiviral compound provided herein is an amount which effectively inhibits fusion of an enveloped virus with the plasma membrane of a cell. In additional aspects, an effective amount of an antiviral compound described herein is an amount which is effective to ameliorate one or more symptoms associated with an enveloped virus infection.

One of ordinary skill in the art can empirically determine effective amounts of antiviral compounds provided herein by routine experimentation.

In some aspects, methods are provided for preventing the spread of a sexually transmitted disease caused by an enveloped virus, such as but not limited to a herpesvirus (e.g., HSV-1 or HSV-2) or HIV, comprising administering an antiviral compound described herein to a subject who is at risk of being exposed to an enveloped virus. In some preferred aspects, the antiviral compound is administered as a topical formulation. In further preferred aspects, the antiviral compound is administered in conjunction with a device, such as but not limited to a condom or other contraceptive device.

In additional aspects, methods are provided for preventing infection due to an intentional exposure to an enveloped virus, for example related to biological warfare or terrorism, wherein the methods comprise administering an antiviral compound described herein to a subject who is at risk of being exposed to an enveloped virus.

Also provided herein are methods for inactivating enveloped viruses in a biological or pharmaceutical preparation, the methods comprising adding an antiviral compound provided herein to the preparation and incubating the mixture for a time sufficient to inactivate enveloped viruses present in the preparation.

In some preferred aspects, the antiviral compounds are substantially inert with respect to the structure and function of macromolecules, cells, tissues, organs and/or other biological structures comprising the preparation. For example, in some aspects, treating biological preparations with the antiviral compounds at a concentration and time sufficient to inactivate enveloped viruses within the preparation does not result in detectable protein denaturation, protein degradation, plasma membrane disruption, cell lysis, or the like.

In additional aspects, biological preparations treated with the antiviral compounds, at a concentration and time sufficient to inactivate enveloped viruses within the preparation, are substantially non-toxic to subjects to whom the preparations are intended for delivery, including but not limited to, human subjects. In some preferred aspects, biological preparations treated with the antiviral compounds are substantially non-toxic to human subjects without the need for further purification or processing.

In some aspects, the biological preparation is a biological sample drawn from a human or animal donor, such as but not limited to, blood, plasma, cerebrospinal fluid, mammary fluid, embryonic fluid, mucus, urine, and the like. For example, in some aspects, blood, tissue, or an organ harvested from a human or animal donor is treated according to methods provided herein to inactivate enveloped viruses, such as but not limited to HIV, prior to transplantation into a human or animal recipient. In some preferred aspects, the treated donor sample is transplanted into the recipient without removing the antiviral compound(s).

In further aspects, the biological preparation comprises cultured cells, tissues, or organs, such as but not limited to stem cells or xenographic tissues intended for transplantation. In additional aspects, the biological preparation comprises cultured host cells for the production of a recombinant protein or other biological product. In some preferred aspects, treating a cellular preparation with an antiviral compound provided herein inactivates enveloped viruses within the preparation without substantially affecting the growth, proliferation, viability, and/or productivity of the cells. In some preferred aspects, the treated cells are used or harvested without removing the antiviral compound(s).

Also provided herein are kits comprising a container housing a an antiviral compound provided herein and instructions for administering the compound to a subject that has been infected or is at risk of infection by an enveloped virus. The instructions may provide for administration as an oral formulation, by inhalation, by topical administration, by intravenous injection and/or by any other suitable means.

In some aspects, kits provided herein optionally further comprise a pharmaceutical preparation vial and a pharmaceutically acceptable diluent, such as physiological saline for diluting a concentrated solution, salt or lyophilized powder formulation of an antiviral compound provided herein.

In some aspects, the kit comprises an inhaler for aerosolized administration to the lungs and/or upper respiratory tract. In further aspects, the kit comprises a device for intranasal administration to the nasal mucosa.

### EXAMPLES

### Example 1

### General Methods

Biologic containment. Infectious materials and animals were handled in maximum containment biosafety level 4 facilities at University of Texas Medical Branch (UTMB) and United States Army Medical Research Institute of Infectious Diseases (USAMRIID). Laboratory personnel wore positive-pressure protective suits (ILC Dover, Frederica, Del.) equipped with high-efficiency particulate air filters and supplied with umbilical-fed air.

Pseudotyped virus production and infection. NiV-F/G and VSV-G pseudotyped VSV viruses were prepared and assayed for infection as previously described (Wolf et al., “A High Throughput Screen for Small Molecule Antagonists of Nipah Virus Infection,” in American Society for Virology, University of Wisconsin at Madison (2006); Negrete et al., Nature, 436:401-5 (2005); Negrete et al., PLoS Pathog, 2:e7 (2006); and Aguilar et al., J Virol, 80:4878-89 (2006), all of which are herein incorporated by reference in their entirety). Unless indicated otherwise, all infections were performed in 1% fetal bovine serum/1×PBS. All pVSV pre-treatments with compound were carried out at 25° C. for 10′. Changes in the pre-treatment temperature (measured at 4° C., 25° C., and 37° C.) and duration of treatment (1 min., 10 min., and 30 min.) had no significant effect on LJ001's antiviral activity (data not shown).

High-throughput screening. HTS was performed on the Chembridge DIVERset® library (Chembridge Corp., San Diego, Calif.). NiV-F/G and VSV-G pseudotyped VSVΔG::rluc viruses were used to infect Vero cells at 90% confluency in 384-well plate format in the presence of small molecules at 10 μM final concentrations, and the viruses were assayed for pseudotyped viral entry as described above.

Viral strains. Vesicular Stomatitis, Indiana; Ebola, Zaire/(ma)Zaire; Marburg, Musoke/Ravn; Junin, Romero; Rift Valley fever, ZH501 and MP-12 (vaccine strain); LaCrosse, Prototype; Omsk Hemorrhagic Fever, Guriev; Russian Spring Summer Encephalitis, Sofiin; Sendai, Enders; Human Parainfluenza Type-3, C-243; HIV-1, JRCSF/YU2; Murine Leukemia, F57; Cowpox, Brighton; Vaccinia, VTF1.1; Adenovirus, Ad5-eGFP; Coxsackie B, eGFP (Feuer et al., J Virol, 76:4430-40 (2002)); Influenza A, WSN H1N1; Nipah, Malaysia; Yellow Fever, Asibi; Hepatitis C, JFH1; West Nile Virus, New York 385-99; Reovirus (Mammalian Orthoreovirus), Type 3 Dearing; Newcastle Disease, rNDV/F3aa-GFP. Poxyiridae stocks likely consisted of infectious single membraned IMVs (intracellular membraned virions) rather than double membraned EEVs (extracellular enveloped virions).

In vitro toxicity assays. Cellular toxicity was assayed using Adenylate Kinase (AK) (Cambrex Corp., East Rutherford, N.J.), Lactate DeHydrogenase (LD) (Takara Bio. Inc., Otsu, Shiga, Japan), and Alamar Blue (AB) (Invitrogen, Carlsbad, Calif.) cytotoxicity assays per manufacturer's instructions.

Cell-cell syncytia assay. Assays of NiV-F/G cell to cell homologous fusion and syncytia were conducted as previously described (Aguilar et al., J Virol, 80:4878-89 (2006); Levroney et al., J Immunol, 175:413-20 (2005)).

Virion purification. Unless otherwise indicated, virus particles were purified through a 20% sucrose cushion for at least 1 h at 110,000×g. For live VSV repurification experiments, viruses were pelleted through a 10% sucrose cushion.

Preparation of LI-series compounds. Compounds were initially resuspended in 100% DMSO (Sigma-Aldrich, St. Louis, Mo.) at a final concentration of 10 mM. LJ-series compounds were prepared by literature methods, e.g., as described herein.

In vitro transcription and translation. Purification of VSV RNP complex and assays of VSV based in vitro transcription, translation, and cap methylation were performed as previously described (Li et al., J Virol, 82:775-84 (2008); Li et al., Proc Natl Acad Sci USA, 103:8493-8 (2006); Li et al., J Virol, 79:13373-84 (2005), all of which are herein incorporated by reference in their entirety).

Manufacture of recombinant liposomes. Recombinant unilammelar liposomes (7:3 molar ratio of PC:Cholesterol) were manufactured by Encapsula Nanosciences, LLC (Nashville, Tenn.). Uniform silica microspheres used for background and signal:noise subtraction during binding assays were purchased from Bangs Laboratories, Inc. (Fisher, Ind.).

R18 assays. Octadecyl rhodamine B chloride (R18) dye was purchased from Molecular Probes (Eugene, Oreg.).

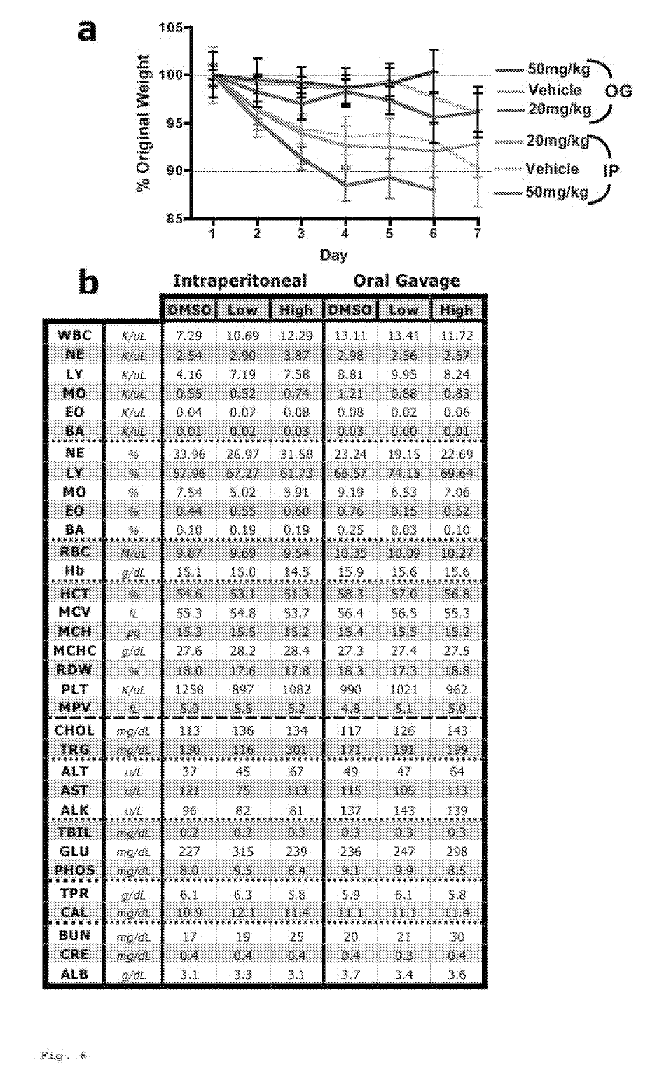

In vivo toxicity assay. Female Balb/c mice were dosed with 20/mg/kg or 50 mg/kg of LJ001 in DMSO by oral gavage or intraperitoneal injections, as described herein. Full toxicology studies (FIG. 6) were performed by Charles River Laboratories (Wilmington, Mass.).

Mass spectrometric analyses of pharmacokinetic serum samples. To the thawed serum samples, methanol (700 μl) was added with the internal standard (²H₅-LJ001, 200 pmol in 20 μl chloroform), and the mixtures were vigorously mixed and centrifuged (20,000×g, 2 min). The supernatants were transferred to HPLC injector vials and dried in a vacuum centrifuge, and dried residues were then redissolved in chloroform (20 μl) to which was sequentially added acetonitrile (120 μl) followed by acetonitrile/water (50/50, 120 μl) containing 0.1% formic acid. The samples were mixed and aliquots of the resulting solutions (200 μl) were injected onto a reverse phase HPLC column (Waters XTerra®, 4.6×100 mm, 3.5 μm particle size) equilibrated in 90% buffer A (0.1% TFA in water)/10% buffer B (0.1% TFA in acetonitrile), and eluted (500 μl/min) with an increasing concentration of acetonitrile (min/% acetonitrile; 0/10, 5/10, 30/100, 33/100, 35/10, 45/10). The effluent was directed into an atmospheric pressure chemical ionization source (probe 450° C., particulate and hydrocarbon depleted air for nebulizing gas) connected to a triple quadrupole mass spectrometer (PE Sciex API III⁺, oriface at 65 volts) operating in the positive ion multiple reaction monitoring tandem mass spectrometric mode in which the collision chamber was flooded with argon gas (collision gas thickness instrumental setting at 100), and the intensity of the parent (protonated molecules)→fragment ions transitions (m/z 328.1→200.1 and 333.1→205.1 for LJ001 and ²H₅-LJ001, respectively) were recorded. LJ001 and ²H₅-LJ001 eluted virtually simultaneously at 29 min. Peak areas were computed using instrument manufacturer supplied software (MacSpec version 3.3), and the amount of drug in each sample was calculated using a standard calibration curve prepared from standard samples containing increasing amounts of LJ001 and a fixed amount of ²H₅-LJ001.

LJ001 produced a negligible signal with electrospray ionization but produced a prominent signal corresponding to the protonated molecule at m/z 328.1 (calculated as 328.0468 Da for C₁₇H₁₄O₂S₂N) during APCI. The penta-deuterated internal standard yielded a corresponding signal at m/z 333.1. During collisionally activated dissociation, both compounds produced numerous fragment ions. The most intense ions at m/z 200.1 and 205.1 for LJ001 and ²H₅-LJ001, respectively, were assigned C₁₂¹H₈OS and C₁₂¹H₃²H₅OS elemental compositions, respectively.

Statistical Analyses. All p values were calculated using an unpaired, two-tailed students' t-test unless indicated otherwise. 95% Confidence Intervals (CI) were calculated using the GraphPad PRISM® regression software.

### Example 2

### Broad Spectrum Antiviral Activity of Arylmethylidene Rhodanine Derivative

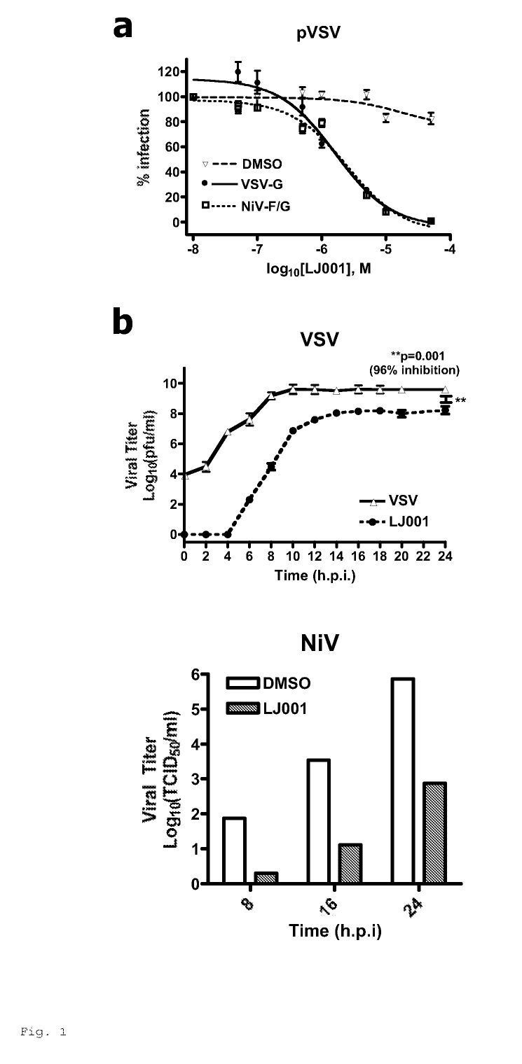

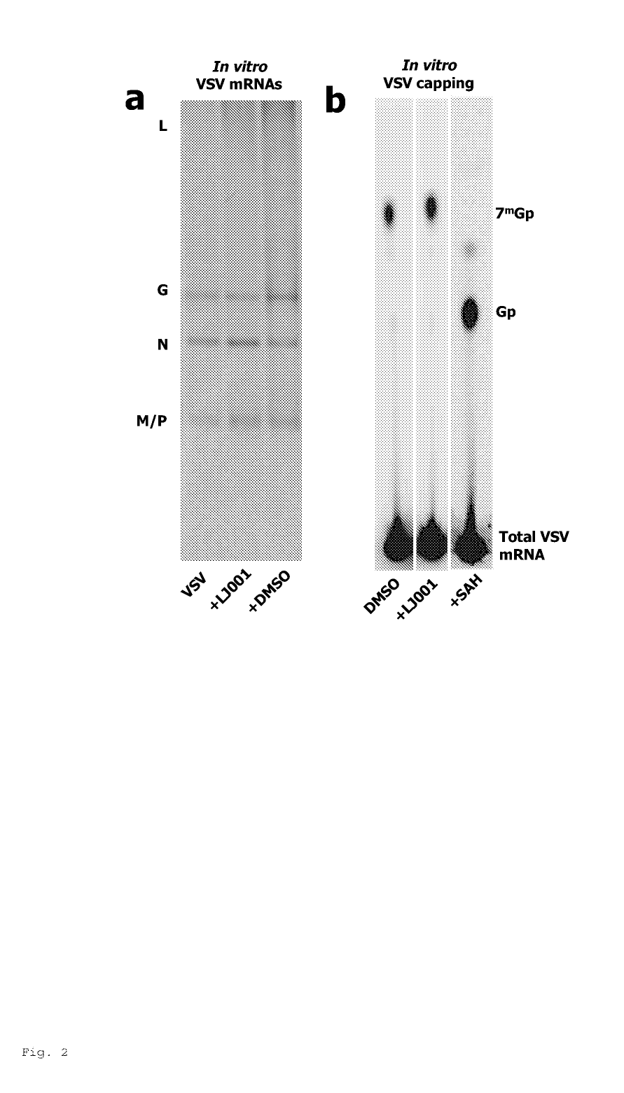

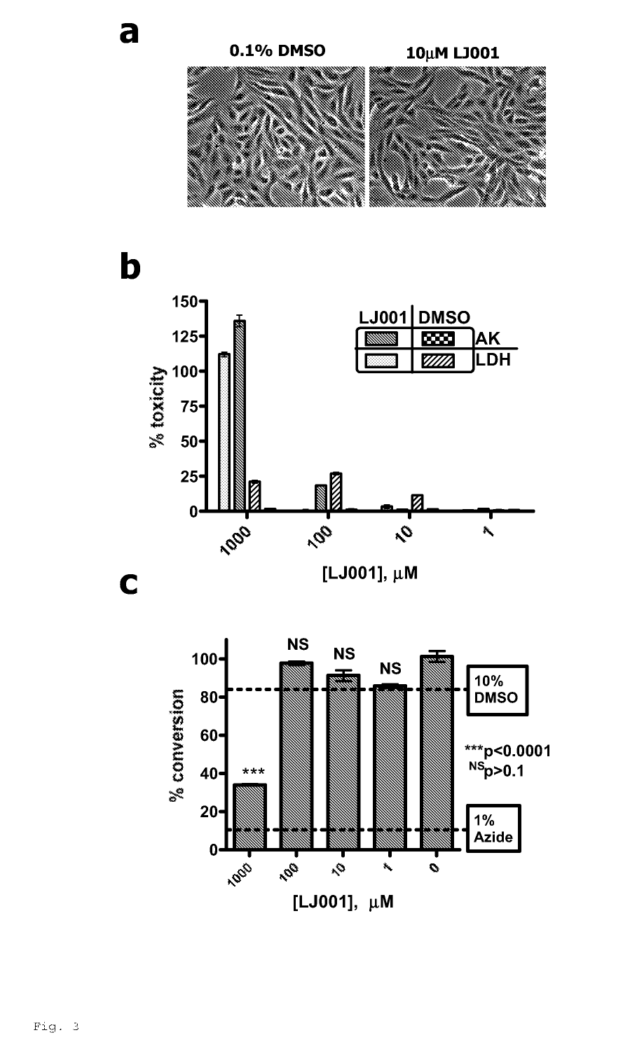

A high-throughput assay (Wolf et al., “A High Throughput Screen for Small Molecule Antagonists of Nipah Virus Infection,” in American Society for Virology (University of Wisconsin at Madison, 2006)) for inhibitors of Nipah virus entry identified an arylmethylidene rhodanine derivative, termed LJ001 ((Z)-3-Allyl-5-(5-phenyl-2-furyl)methylene-4-oxothiazolidine-2-thione). LJ100 inhibited reporter virus entry via both Nipah virus envelope (NiV-F/G) and Vesicular Stomatitis virus envelope proteins (VSV-G) pseudotyped onto VSV-luciferase reporter virus (VSVΔG::Renilla luciferase) (FIG. 1a), indicating that the inhibitory effect is not specific to the viral envelope proteins. This was confirmed by LJ001's ability to inhibit infection and infectious spread of live NiV and VSV in vitro (FIG. 1b). Viral transcription, mRNA production, and mRNA capping of VSV were unaffected by 10 μM LJ001 in an in vitro assay of VSV-based transcription independent of viral entry (FIG. 2).

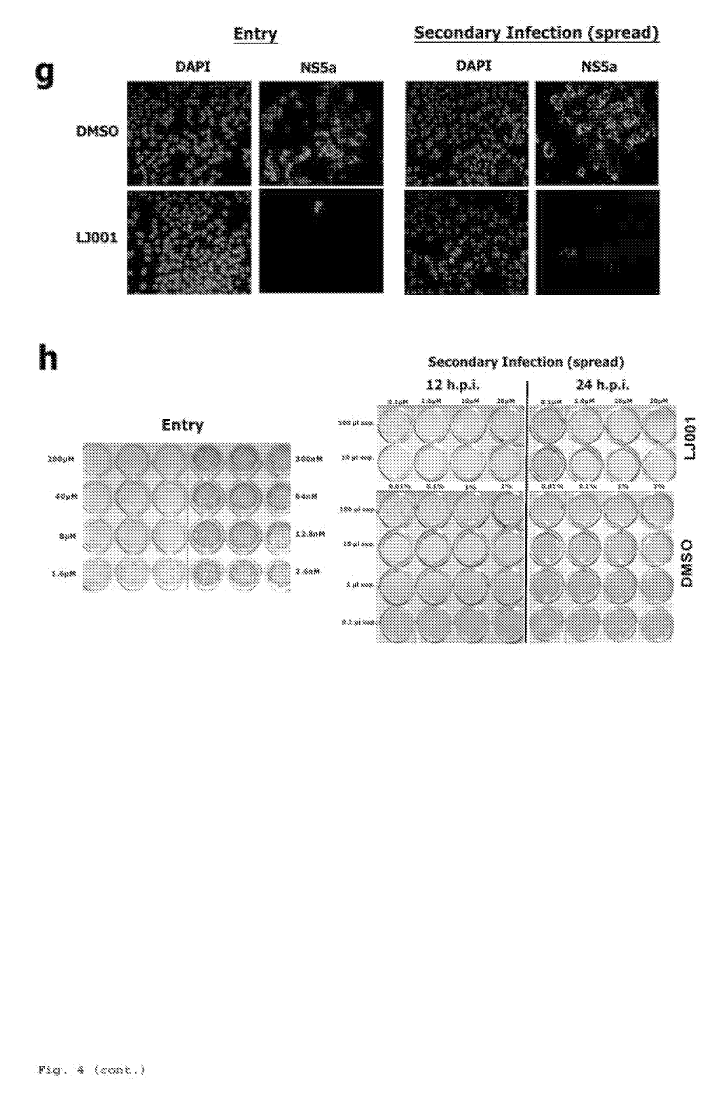

LJ001 exhibited a broad-spectrum antiviral capability, inhibiting entry, and sometimes infectious spread, of a wide variety of lipid-enveloped viruses, including HIV, HCV, and numerous highly pathogenic Category A-C “priority pathogens” without affecting non-enveloped viruses (Table 1 and FIG. 4). LJ001 demonstrated roughly similar efficacy amongst the enveloped viruses tested, despite the different target cell types, viruses, and measures of infectivity used in the assays. The results suggest a common mechanism of inhibition, likely targeting an invariant component of enveloped viruses.

TABLE 1
LJ001 inhibits a variety of enveloped, but not non-enveloped, viruses in vitro.

| | NIAID | | Genome | Enveloped | |
|---|---|---|---|---|---|
| Virus | Category | Family | Type | (Yes/No) | Activity |
| Ebola | A | Filoviridae | ssRNA(−) | Y | ++ |
| Marburg | A | Filoviridae | ssRNA(−) | Y | ++ |
| Influenza A | A | Orthomyxoviridae | ssRNA(−) | Y | +++ |
| Junín | A | Arenaviridae | ssRNA(−) | Y | ++ |
| Rift Valley Fever | A | Bunyaviridae | ssRNA(−) | Y | +++ |
| LaCrosse | B | Bunyaviridae | ssRNA(−) | Y | +++ |
| Nipah | C | Paramyxoviridae | ssRNA(−) | Y | ++ |
| Omsk | C | Flaviviridae | ssRNA(+) | Y | ++ |
| RSSE | C | Flaviviridae | ssRNA(+) | Y | ++ |
| PIV-5 | — | Paramyxoviridae | ssRNA(−) | Y | ++ |
| HPIV-3 | — | Paramyxoviridae | ssRNA(−) | Y | ++ |
| NDV* | — | Paramyxoviridae | ssRNA(−) | Y | ++ |
| HIV-1* | — | Retroviridae | ssRNA(−)RT | Y | ++ |
| Murine Leukemia | — | Retroviridae | ssRNA(−)RT | Y | ++ |
| Yellow Fever | — | Flaviviridae | ssRNA(+) | Y | +++ |
| Hepatitis C Virus | — | Flaviviridae | ssRNA(+) | Y | +++ |
| West Nile Virus | — | Flaviviridae | ssRNA(+) | Y | +++ |
| Vesicular | — | Rhabdoviridae | ssRNA(−) | Y | ++ |
| Cowpox | — | Poxviridae | dsDNA | Y | + |
| Vaccinia | — | Poxviridae | dsDNA | Y | ++ |
| Adenovirus** | — | Adenoviridae | dsDNA | N | − |
| Coxsackie B** | — | Picornaviridae | ssRNA(+) | N | − |
| Reovirus | — | Reoviridae | dsRNA | N | − |

Virus infections were performed using varying concentrations of LJ001 and inhibition was determined by measuring viral titers by standard plaque assays or TCID₅₀, unless indicated otherwise (*= qPCR, **= Flow cytometric analysis of recombinant GFP expressing virus).
+++, IC₅₀ < 0.5 μM;
++, 0.5 μM < IC₅₀ < 1 μM;
+, 1 μM < IC₅₀ < 5 μM;
−, no significant inhibition at >10 μM.
Raw data for representative viruses are shown in FIG. 4.

### Example 3

### In Vitro and In Vivo Toxicity

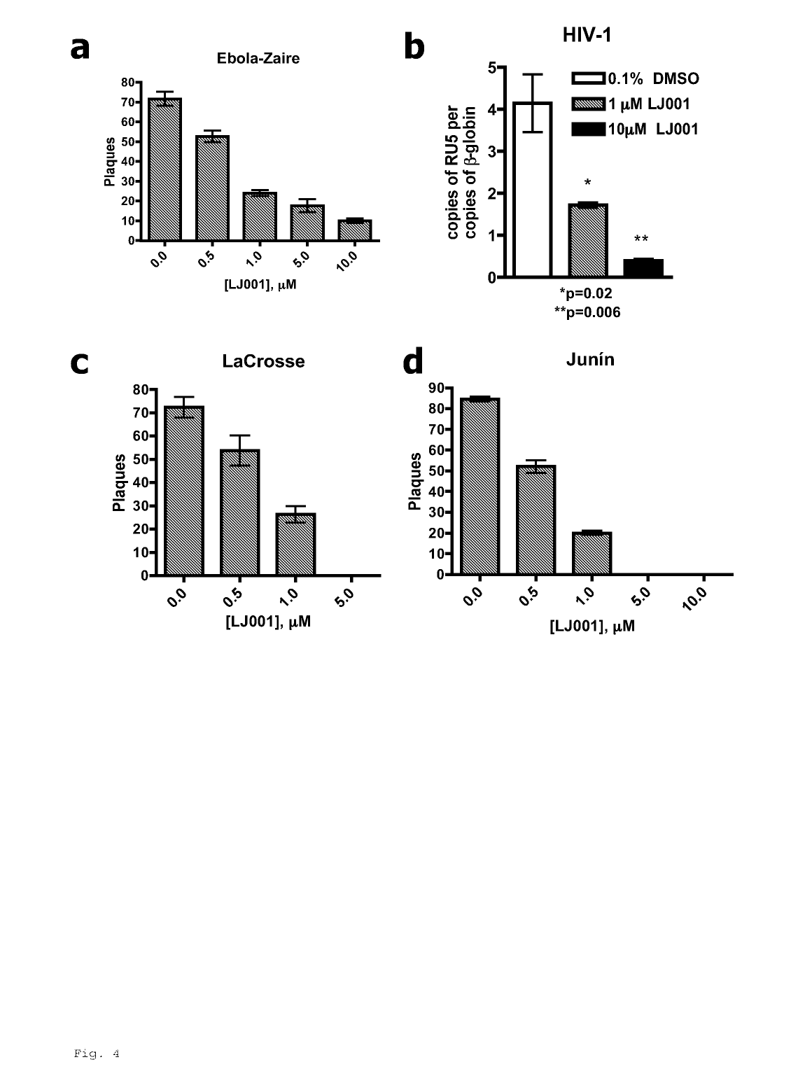

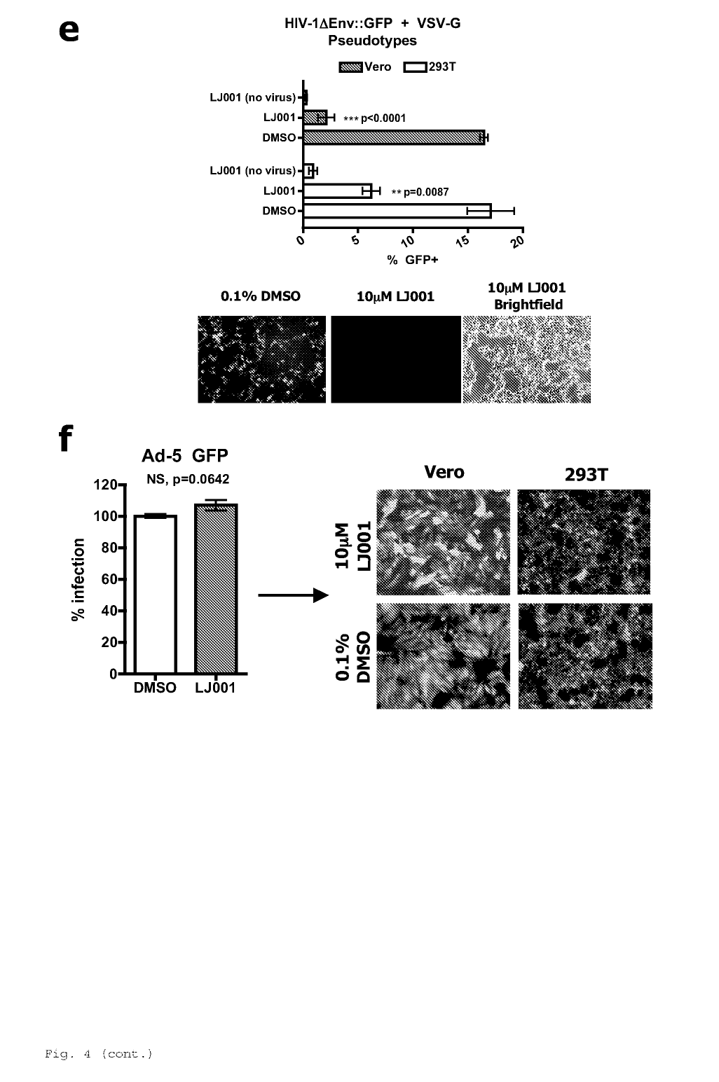

To rule out non-specific cytotoxic effects as the basis for the antiviral activity of LJ001, Vero cells were repeatedly passaged in the presence of 10 μM LJ001 (˜10 times the IC₅₀) over a period of 4 days. No overt deficiencies were observed with regards to cell division, changes in morphology, or other gross signs of toxicity (FIG. 3a). In addition to Vero cells, various primary cells and other cell lines were exposed to various concentrations of LJ001 for 1 hour, mimicking conditions during infection, and then subjected to adenylate kinase (AK) and lactate dehydrogenase (LD) enzyme release assays to test for cellular toxicity. LJ001 showed little to no toxic effects compared to DMSO (vehicle control) at concentrations up to 10 μM (FIG. 3b). While data are shown for Vero cells (used for almost all virus infections), other common laboratory cell lines such as MDCK, HeLa, 293T, and CHO cells were also tested along with primary microvascular endothelial cells and PBMCs (data not shown). Finally, an Alamar Blue uptake assay (Al-Nasiry et al., Hum Reprod, 22:1304-9 (2007)) indicated no effect on active cell metabolism in LJ001-treated Vero cells (FIG. 3c). Although some variance in the degree of cytotoxicity was observed amongst different cell lines with different passage histories, no overt cellular toxicity could be detected in cells at concentrations that significantly inhibited virus infection (unpublished observations).

To test for LJ001 toxicity in vivo, female adult BALB/c mice were dosed orally (OG) and intraperitoneally (IP) with 20 mg/kg and 50 mg/kg LJ001 (50 μl dose in 100% DMSO) daily for 7 days (n=3/group, total n=18). Although slight weight loss (<10%) occurred in the IP dose group, effects were comparable to the vehicle control group (FIG. 6). At the end of the dosing regimens, the mice were sacrificed and complete blood chemistry panels, cell counts, and organ toxicology tests were conducted. Liver function tests (ALT, AST, ALK, TBIL), kidney function tests (ALB, BUN, CRE), and serum electrolytes (Ca²⁺, Na⁺, K⁺, Cl⁻) were all normal and equivalent to the vehicle control mice in both 20 mg/kg and 50 mg/kg dosing groups (FIG. 6). Complete blood counts with differential showed no difference relative to the vehicle control group, and hematocrit and hemoglobin levels were also normal (FIG. 6). Serum metabolites (glucose, phosphate, triglycerides) were normal except for a statistically significant elevation in serum cholesterol levels in the treated vs. vehicle control group (FIG. 6b-c).

### Example 4

### Antiviral Mechanism

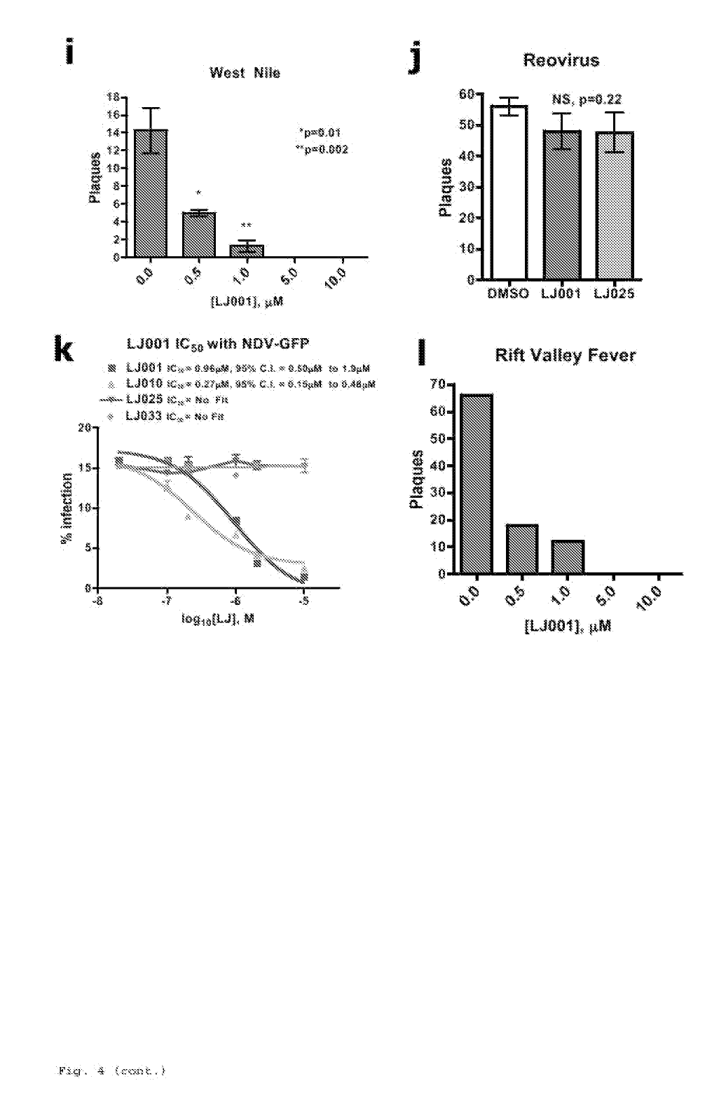

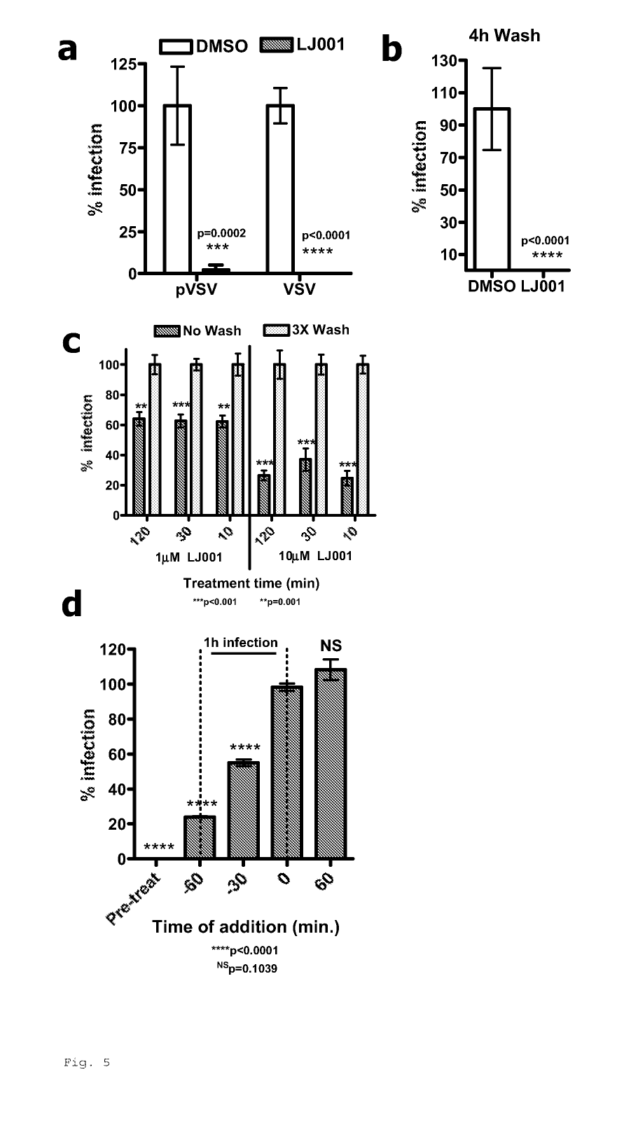

The mechanism by which LJ001 inhibits productive virus infection without imparting overt toxicity in vivo or in vitro toxicity to the host was investigated by pretreating live HPIV-3 (data not shown), live VSV, and NiV-envelope pseudotyped onto VSV (NiV-pVSV) with LJ001 followed by washing, repurification, and infection of cells with the repurified viruses (FIG. 5a). Viruses treated in this manner were non-infectious. The repurified virus was then washed with 6 mL PBS for 4 h and subjected to a secondary repurification and re-infection of cells (FIG. 5b). The virus again remained non-infectious, suggesting that LJ001 acts viruses in an irreversible manner.

To investigate the specificity of LJ001 for the virus relative to host cells, target cells were pre-treated with 10 μM LJ001, washed to remove residual compound, and infected with pVSV (FIG. 5c) or VSV-G pseudotyped HIV-1 (data not shown). Washing the cells reversed the inhibitory effect of LJ001, suggesting that LJ001 does not act on host cells.

The temporal aspects of LJ001 antiviral activity were investigated by conducting time-of-addition experiments. As shown in FIG. 5d, the inhibitory effect of LJ100 was apparent only when the compound was added before or during, but not after, the viral infection period. The results indicate that LJ001 acts on viruses before entry but not at the level of viral transcription, translation, or replication.

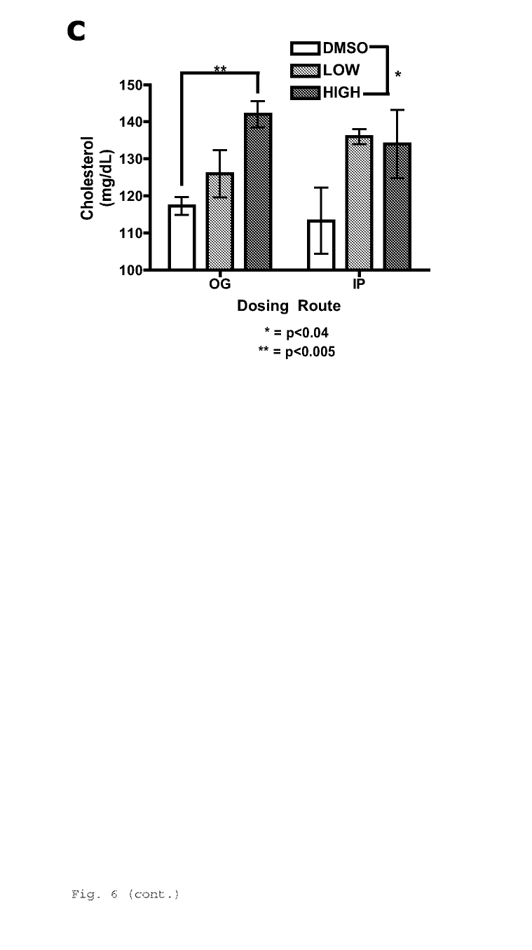

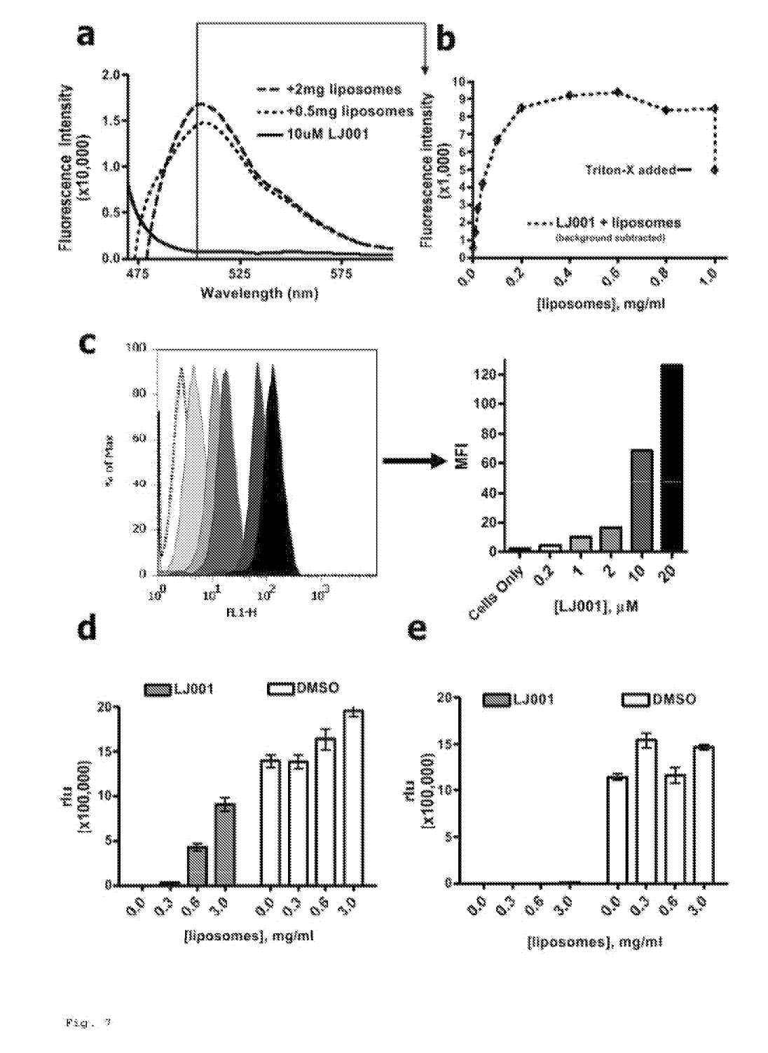

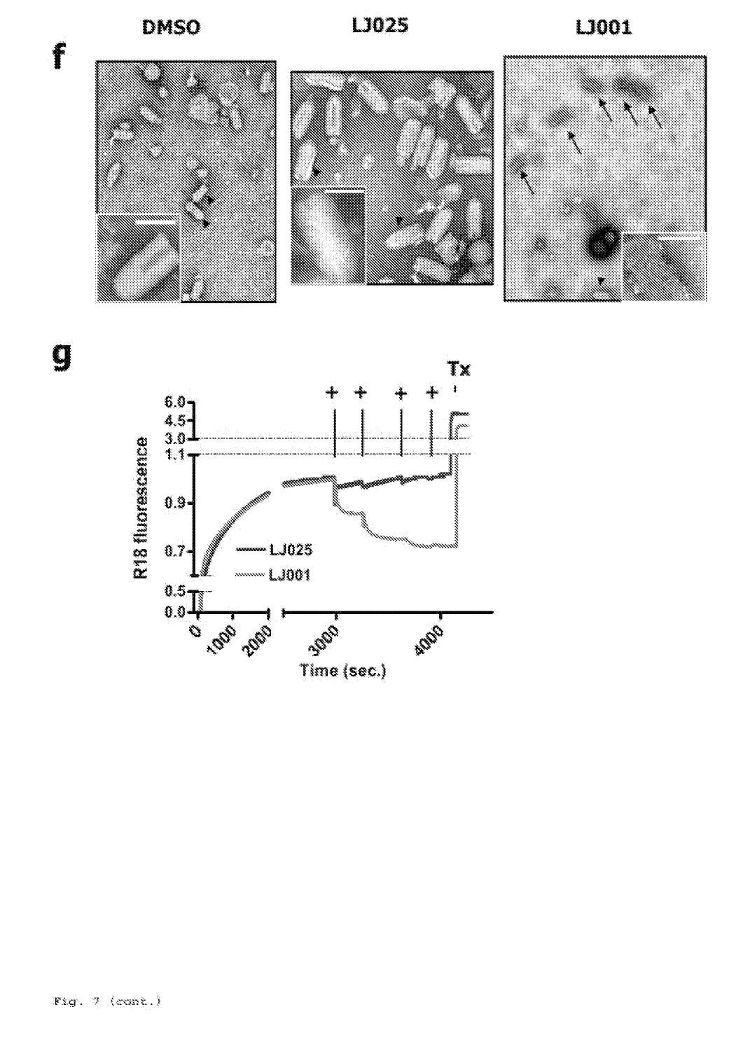

Since LJ001 appears to act selectively against a viral component common to all enveloped viruses, it was hypothesized that LJ001 targets the viral lipid membrane. The ability of LJ001 to bind lipid membranes was investigated by measuring LJ001 binding to manufactured liposomes that biochemically mimic cellular lipid bilayers. Binding was detected using a fluorescence intensity-based membrane intercalation assay based on LJ001's inherent fluorescent properties. LJ001 has minimal fluorescence in aqueous solvent alone but fluoresces strongly upon intercalation into lipid membranes. Thus, LJ001 exhibited increasing fluorescence in the presence of increasing concentrations of liposomes, but did not fluoresce in the presence of similarly sized hydrophilic silica beads (FIG. 7a and FIG. 8a). The intercalation of LJ001 into lipid membranes was specific and saturable, and the interaction was dependent on intact liposomal membranes, as introduction of the detergent Triton X-100 resulted in a loss of fluorescence (FIG. 7b).

Since viral membranes are derived from host cell membranes, a potential concern with the use of small molecule inhibitors that target and disrupt viral lipid membranes is the possibility of activity against host cell membranes. To investigate this possibility, Vero cells were treated with various concentrations of LJ001 and were then fixed and analyzed for binding using flow cytometry (FIG. 7c). LJ001 intercalated into cellular membranes, resulting in a dose-dependent increase in fluorescence. Thus, LJ001 binds to both viral and host cell membranes, and yet clearly acts only against viruses and not against host cells (FIG. 5).

The selective activity of LJ001 may be related to the underlying biophysical and physiological differences between viral and cellular membranes. Mammalian cell membranes are “biogenic” in that they are capable, through poorly understood mechanisms, of rapidly (e.g., within seconds) detecting and repairing plasma membrane lesions. Additionally, host cells continuously metabolize and recycle fatty acids and other membrane components in order to replenish and maintain healthy plasma membranes (McMahon and Gallop, Nature, 438:590-6 (2005); Kent, Annu Rev Biochem, 64:315-43 (1995); Koval and Pagano, J Cell Biol, 108:2169-81 (1989); Sleight and Pagano, J Cell Biol, 99:742-51 (1984); Steinman et al., J Cell Biol, 96:1-27 (1983)). Even though viral membranes are derived from host cell membranes, virions inherently lack the ability of cell membranes to actively produce/recycle lipids or repair damaged membranes, making viruses particularly susceptible to membrane disruption (McNeil and Steinhardt, Annu Rev Cell Dev Biol, 19:697-731 (2003); McNeil and Terasaki, Nat Cell Biol 3:E124-9 (2001); Meldolesi, J Cell Mol Med, 7:197-203 (2003)). Thus, arylmethylidene rhodanine derivatives, such as LJ001, may exploit physiological differences between viral membranes and biogenic cell membranes.

To determine if the inhibitory effect of LJ001 during infection could be reversed by the addition of liposomes, cells were infected with NiV-pVSV in the presence of a fixed concentration of LJ001 and increasing liposome concentrations (FIG. 7d). The liposomes competed off virus infection when the assay was conducted by simultaneously subjecting both the virus and liposomes to LJ001. However, if the viral particles were pre-exposed to LJ001 before adding the mixture to liposomes, the presence of excess liposomes is no longer able to rescue viral infection (FIG. 7e). When 10 μM LJ001 was pre-incubated with a saturating amount of liposomes, the LJ001-saturated liposomes had no effect on viral infection, regardless of virus particle incubation times (FIG. 8c).

To assess if membrane curvature influences LJ001 antiviral activity, liposome binding and infection-competition assays, as in FIG. 7a and FIG. 7d, were performed using differentially sized liposomes (ranging from 50 nm to 600 nm). Liposome size had no effect on LJ001 binding or reversal of inhibition (FIG. 8d-e), which is consistent with the ability of LJ001 to inhibit infection by a wide range of viruses with sizes, shapes and morphologies.

The effect of arylmethylidene rhodanine derivatives on the biophysical properties of viral lipid membranes was further investigated using the lipophilic dye octadecyl rhodamine B chloride (R18), which exhibits increased fluorescence when integrated into lipid bilayers (Ohki et al., Biochemistry, 37:7496-503 (1998); Connolly et al., Virology, 355:203-12 (2006)). R18 can self-quench at high densities and its fluorescent dequenching is often used as a measurement of lipid mixing during virus-cell fusion. Liposomes were loaded with R18 and realtime uptake and diffusion into the liposome bilayers was detected as an increase in fluorescence. The liposomes were then treated with increasing concentrations of LJ001 or LJ025 (FIG. 7g). LJ001, but not LJ025, caused a saturable decrease in fluorescence. The LJ001-induced decrease in R18 fluorescence could be due, e.g., to increased R18 aggregation and quenching within the membrane, or release of R18 from the lipid membrane leading to sub-saturating fluorescence. In either case, LJ001 but not LJ025 clearly leads to quenching of the R18 signal, indicating that LJ001 affects the lipid dynamics or biophysical properties of membranes in a manner different from LJ025.

### Example 5

### Effect of Arylmethylidene Rhodanine Derivatives on Virion Structure

To further investigate the effects of arylmethylidene rhodanine derivatives on virus particles, DMSO-, LJ001- and LJ025-treated pVSV particles were imaged via electron microscopy. LJ001 induced a significant distortion of the viral membrane (FIG. 7f), albeit at higher concentrations than needed for viral neutralization. The obvious presence of a negative stain in the interior of virions treated with LJ001, but not LJ025 or DMSO, suggests that the membranes of LJ001-treated virions were permeabilized to some degree. Electron microscopy experiments conducted with NDV showed similar results (FIG. 8f).

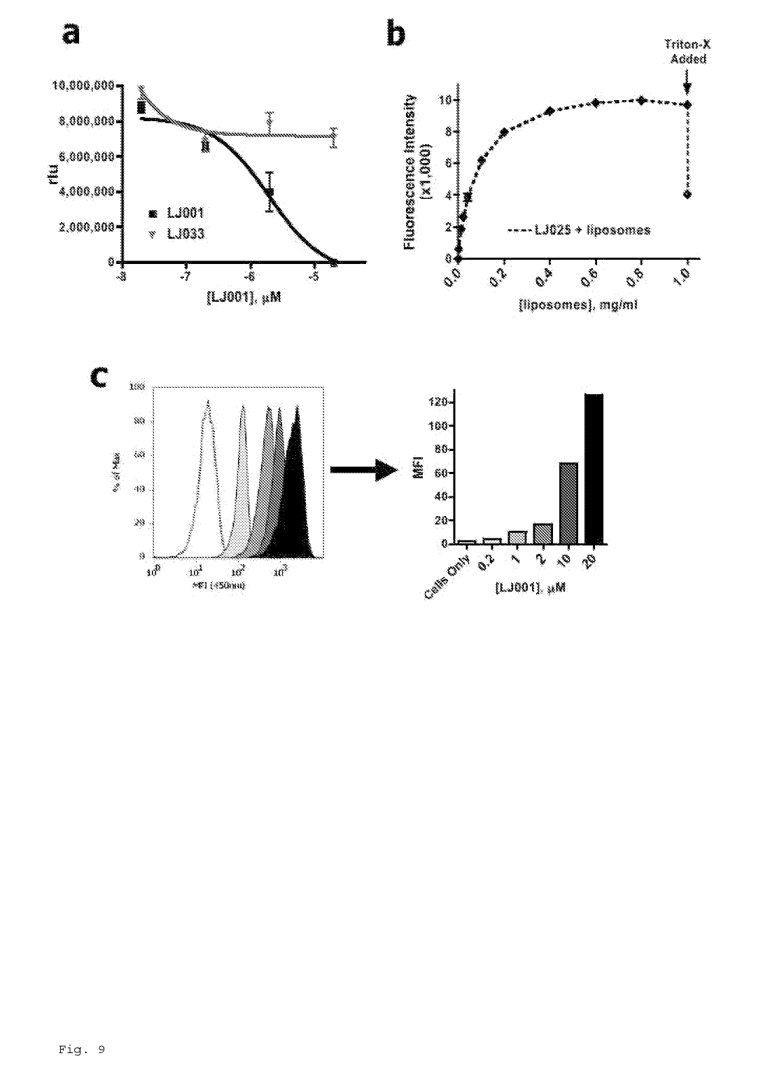

The effect of arylmethylidene rhodanine derivatives on virus particle structure and function was further assessed by treating virus particles with LJ001 and analyzing the content and infectivity of the particles by western blotting and plaque assays, respectively. To underscore the broad-spectrum activity arylmethylidene rhodanine derivatives, the experiments were conducted with Rift Valley fever virus (RVFV MP-12), which is another highly pathogenic Category A priority pathogen. RVFV MP-12 treated with LJ001 or DMSO was repurified via banding through a density gradient, and fractions were processed for either Western blotting or infectivity determination by plaque assays. FIG. 10a shows that the RVFV envelope and nucleocapsid-proteins banded at the same buoyant density regardless of LJ001 treatment, although there may have been a slight loss of membrane (GN/GC) or nucleocapsid (N) proteins in the LJ001 treated samples. Vehicle control (DMSO) treated fractions remained fully infectious; however, those fractions treated with LJ001 were completely non-infectious despite the obvious presence of intact virions in lane 8-9 (FIG. 10b). The same assay repeated with pVSV produced similar results (data not shown).

Thus, the results indicate that envelope glycoproteins of LJ001-treated RVFV and pVSV remain associated with the viruses, although the viruses themselves remain noninfectious (FIG. 10a-b). To determine whether arylmethylidene rhodanine derivatives affect receptor binding envelope proteins, viruses were incubated with CHO cells stably expressing the NiV receptor, ephrinB2 (Negrete et al., J Virol, 81(19):10804-14 (2007); Negrete et al., Nature, 436:401-5 (2005); Negrete et al., PLoS Pathog, 2:e7 (2006)), and virus binding was assayed in the presence or absence of LJ001 with anti-NiV-F polyclonal antibodies (FIG. 10c-d) (Negrete et al., Nature, 436:401-5 (2005); Aguilar et al., J Virol, 80:4878-89 (2006)). The ability of soluble ephrinB2 to compete for virus-cell binding demonstrates the specificity of the assay (FIG. 10c-d). Since the viral envelope proteins still bound cognate cell surface receptors, the assays indicated that arylmethylidene rhodanine derivatives arrest the viral entry process at a step after virus binding.

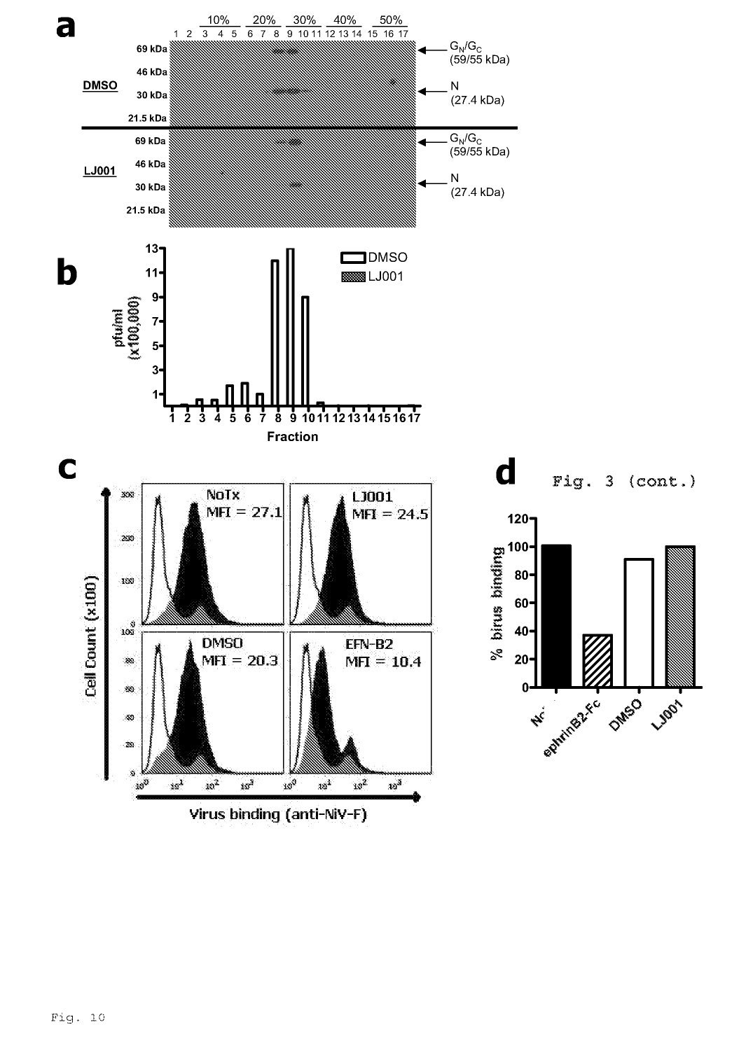

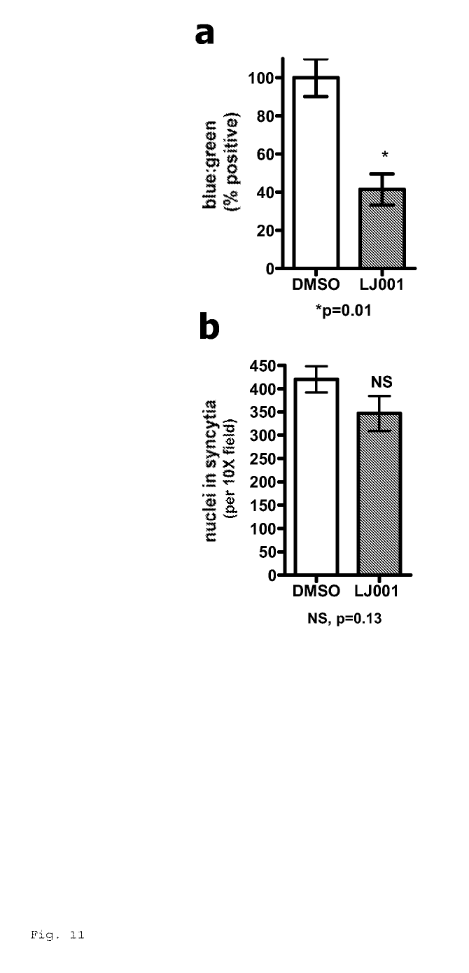

Since the viral envelope appears deformed yet functionally intact, the effect of arylmethylidene rhodanine derivatives on virus-cell fusion and delivery of virion contents to the cytosol was investigated using a newly developed NiV matrix based virus-like particle (VLP) entry assay where virus-cell fusion and entry were monitored by cytosolic delivery of a reporter protein fused to the NiV matrix protein, circumventing the need for viral transcription or translation (Cavrois et al., Nat Biotechnol, 20:1151-4 (2002); Cavrois et al., Methods Mol Biol, 263:333-44 (2004); Cavrois et al., Virology, 328:36-44 (2004)). FIG. 11a shows that LJ001 inhibited cytoplasmic delivery of the beta-lactamase matrix fusion protein, suggesting that arylmethylidene rhodanine derivatives act prior to viral entry and completion of virus-cell fusion.

The effect of arylmethylidene rhodanine derivatives on cell-cell fusion using a synctia assay. Transfection of NiV envelope glycoprotein expression vectors into permissive cells can result in formation of giant multinucleated syncytia from envelope protein-mediated cell:cell fusion in a manner homologous to virus-cell fusion (Negrete et al., Nature, 436:401-5 (2005); Aguilar et al., J Virol, 80:4878-89 (2006); Aguilar et al., Virology, 81:4520-32 (2007); Levroney et al., J Immunol, 175:413-20 (2005); Schowalter et al., Virology, 350:323-34 (2006)). LJ001 did not inhibit NiV envelope mediated cell-cell fusion (FIG. 11b) while it clearly inhibited virus-cell fusion of NiV-pVSV (FIG. 11a and FIG. 1a) and many other enveloped viruses (Table 1). The results underscore the fundamental differences between virus-cell and cell-cell fusion and provide additional evidence that arylmethylidene rhodanine derivatives act by exploiting biophysical and/or physiological differences between virus and host cell membranes.

### Example 6

### Synthesis of Arylmethylidene Rhodanine Derivatives

General Procedure. Materials were obtained from commercial suppliers and were used without further purification. Air or moisture sensitive reactions were conducted under an argon atmosphere using oven-dried glassware and standard syringe/septa techniques. The reactions were monitored via silica gel TLC using UV light (254 nm) followed by visualization with a p-anisaldehyde or ninhydrin staining solution. Column chromatography was performed on silica gel 60. ¹H NMR spectra were measured at 400 MHz in CDCl₃ unless stated otherwise and data were reported as follows in ppm (δ) from the internal standard (TMS, 0.0 ppm): chemical shift (multiplicity, integration, coupling constant in Hz.).

### General Procedure for the Synthesis of 3-Alkyl-4-oxothiazolidine-2-thiones (formula I), 5a-e

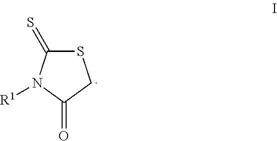

To a solution of the alkyl isothiocyanate, 3, e.g., ethyl isothiocyanate 3b (87.0 μL, 1.0 mmol) in 3.0 mL of dichloromethane was added methyl thioglycolate 4 (89.0 μL, 1.0 mmol) and triethylamine (138.0 μL 1.0 mmol). After the solution was stirred at 25° C. for 2 h, it was treated with 1.0 mL water. The organic layer was washed twice with 1 mL water and dried over NaSO₄. Removal of the solvent gave 150.0 mg (93%) of 3-ethylrhodamine 5b which was used directly in the next step. The 3-alkyl-4-oxothiazolidine-2-thiones are generally known in the art and many are commercially available (for general preparation, see Condon et al., R. Org. Prep. Proc. Int., 6(3):7-43 (1974); and Drobnica et al., E. Chem. Zvesti, 26:538-42 (1972)).

Yields for 5:
3-Allyl-4-oxothiazolidine-2-thione, 5a R═CH₂—CH═CH₂, 90%
3-Ethyl-4-oxothiazolidine-2-thione, 5b R=Et, 93%
3-Propyl-4-oxothiazolidine-2-thione, 5c, R═Pr, 83%
3-Benzyl-4-oxothiazolidine-2-thione, 5d, R═CH₂-Ph, 90%
3-Methyl-4-oxothiazolidine-2-thione, 5e, R=Me, 85%

### General Procedure for the Synthesis of (Z)-3-Alkyl-5-arylmethylene-4-oxothiazolidine-2-thione (formula II), 1

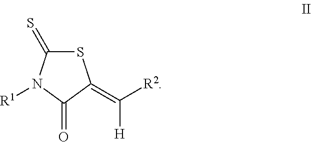

To a solution of the 3-alkylrhodanine 5, e.g., 3-ethylrhodanine 5b (80.0 mg, 0.5 mmol), and sodium acetate (205.0 mg, 2.5 mmol) dissolved in 3.0 mL acetic acid was added 5-phenyl-2-furaldehyde 6 (86 mg, 0.5 mmol). The solution was stirred at 135° C. overnight and diluted with 30.0 mL of ethyl acetate after being cooled to 25° C. The combined organic layer was washed with water (2×20 mL), satd. NaHCO₃ (2×20 mL), and again with water (2×20 mL). After being dried over sodium sulfate, the solvent was removed to give 145.0 mg of crude product 1b.

### (Z)-3-Ethyl-5-(5-phenyl-2-furyl)methylene-4-oxothiazolidine-2-thione, 1b, LJ002, 76%

¹H NMR 7.79 (2H, d, J=8.0 Hz), 7.47 (3H, m), 7.38 (1H, m), 6.94 (1H, d, J=3.6 Hz), 6.86 (1H, d, J=3.6 Hz), 4.20 (2H, q, J=7.2 Hz), 1.30 (3H, t, J=7.2 Hz); ¹³C NMR 194.26, 167.33, 158.87, 149.44, 129.27, 129.14, 128.96, 124.73, 121.35, 120.06, 117.79, 108.92, 39.76, 12.34.

### (Z)-3-Allyl-5-(5-phenyl-2-furyl)methylene-4-oxothiazolidine-2-thione, 1a, LJ001, 80%

¹H NMR 7.91 (2H, d, J=7.8 Hz), 7.59 (3H, m), 7.50 (1H, m), 7.06 (1H, d, J=3.6 Hz), 6.98 (1H, d, J=3.6 Hz), 6.00 (1H, m), 5.38 (2H, m), 4.87 (2H, d, J=4.8 Hz). ¹³C NMR 194.07, 157.15, 158.94, 149.39, 129.75, 129.30, 129.14, 128.90, 124.73, 121.51, 119.76, 119.18, 118.00, 108.96, 46.38.

### (Z)-3-Propyl-5-(5-phenyl-2-furyl)methylene-4-oxothiazolidine-2-thione, 1c, LJ003, 85%

¹H NMR 7.81 (2H, d, J=8.0 Hz), 7.48 (3H, m), 7.40 (1H, m), 6.95 (1H, d, J=3.6 Hz), 6.87 (1H, d, J=3.6 Hz), 4.10 (2H, t, J=6.4 Hz), 1.76 (2H, m), 1.00 (3H, t, J=7.6 Hz); ¹³C NMR 194.59, 167.60, 158.85, 149.46, 129.27, 129.14, 128.96, 124.73, 121.31, 119.99, 117.79, 108.91, 46.00, 20.49, 11.26.

### (Z)-3-Benzyl-5-(5-phenyl-2-furyl)methylene-4-oxothiazolidine-2-thione, 1d, LJ004, 83%

¹H NMR 7.80 (2H, d, J=8.0 Hz), 7.50 (5H, m), 7.40 (1H, m), 7.32 (3H, m), 6.95 (1H, d, J=3.6 Hz), 6.87 (1H, d, J=3.6 Hz), 5.34 (2H, s); ¹³C NMR 194.33, 167.57, 159.00, 149.41, 135.00, 129.32, 129.16, 128.92, 128.58, 128.06, 124.75, 121.56, 119.76, 118.11, 108.97, 47.51 (one carbon unresolved).

### (Z)-3-Methyl-5-(5-phenyl-2-furyl)methylene-4-oxothiazolidine-2-thione, 1e, LJ005, 81%

¹H NMR 7.81 (2H, d, J=8.0 Hz), 7.48 (3H, m), 7.40 (1H, m), 6.95 (1H, d, J=3.6 Hz), 6.87 (1H, d, J=3.6 Hz), 3.54 (3H, s); ¹³C NMR 194.28, 167.21, 158.88, 149.39, 129.32, 129.15, 129.00, 128.94, 124.76, 121.52, 120.07, 118.05, 108.95, 31.24.

### (Z)-3-ethyl-5-((5-methylfuran-2-yl)methylene)-2-thioxothiazolidin-4-one, 1m, LJ013, 77%

¹H NMR 7.40 (1H, S), 6.76 (1H, d, J=3.6 Hz), 6.22 (1H, d, J=3.6 Hz), 4.19 (2H, q, J=7.2H), 2.43 (3H, S), 1.29 (3H, t, J=7.2 Hz); ¹³C NMR 194.4, 167.40, 158.55, 148.83, 120.61, 119.10, 118.35, 110.40, 39.64, 14.28, 12.32.

### (Z)-3-ethyl-5-(furan-2-ylmethylene)-2-thioxothiazolidin-4-one, 1n, LJ014, 75%

¹H NMR 7.70 (1H, d, J=1.5 Hz), 7.53 (1H, S), 6.83 (1H, d, J=3.6 Hz), 6.58 (1H, dd, J=1.5, 3.6), 4.13 (2H, q, J=7.2H), 1.24 (3H, t, J=7.2 Hz); ¹³C NMR 194.32, 167.37, 150.17, 147.00, 121.19, 118.52, 118.15, 113.50, 39.69, 12.30.

### (Z)-3-ethyl-5-((5-(2-nitrophenyl)furan-2-yl)methylene)-2-thioxothiazolidin-4-one, 1o, LJ015, 81%

¹H NMR 7.87 (1H, d, J=1.2 Hz), 7.85 (1H, d, J=1.2 Hz), J=3.6 Hz), 4.20 (2H, q, J=7.2H), 1.27 (3H, t, J=7.2 Hz); ¹³C NMR 193.74, 167.23, 152.39, 150.77, 147.61, 132.51, 129.58, 129.08, 124.43, 122.78, 122.27, 120.30, 117.08, 113.38, 39.79, 12.31.

### General Procedure for the Synthesis of 5-Arylfuran-2-carboxaldehyde, 6

5-Bromofuran-2-carboxaldehyde 7 (175.0 mg, 1.0 mmol) and the arylboronic acid 8, e.g., 3-fluorophenylboronic acid 8g (140.0 mg, 1.0 mmol), were dissolved in a mixture of 10.0 mL toluene and 4.0 mL ethanol. Tetrakis(triphenylphosphinepalladium) (0) (33.0 mg) was added, followed by addition of 10.0 mL of satd. potassium carbonate. The mixture was then heated to reflux for 3 h. Water was added and the mixture was extracted with dichloromethane (2×20 mL). The combined organic layer was dried with sodium sulfate and the solvent was removed. The residue was purified by chromatography on silica gel eluting with hexane/ethyl acetate (5:2) to afford 158.0 mg (83%) of the aldehyde 6g.

6f, R¹═H, R²═Cl, 92%

6g, R¹═H, R²═F, 83%

6h, R¹═F, R²═H, 87%

61, R¹═Cl, R²═H, 90%

6j, R¹═OMe, R²═H, 83%

6k, R¹═H, R²═OMe, 86%

6l, R¹═CF₃, R²═H, 86%

### General Procedure for the Synthesis of (Z)-3-Alkyl-5-(5-aryl-2-furyl)methylene-4-oxothiazolidine-2-thiones (formula III), 2

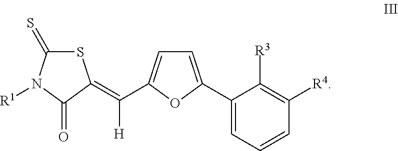

For these compounds, the same procedure was followed as for the synthesis of 1b.

### (Z)-3-Ethyl-5-[5-(3-chlorophenyl)-2-furyl]methylene-4-oxothiazolidine-2-thione, 1f, R¹═H, R²═Cl, LJ006, 73%

¹H NMR 7.70 (2H, d, J=8.0 Hz), 7.48 (1H, s), 7.37 (1H, m), 7.34 (1H, m), 6.95 (1H, d, J=3.6 Hz), 6.88 (1H, d, J=3.6 Hz), 4.20 (2H, q, J=7.2 Hz), 1.28 (3H, t, J=7.2 Hz); ¹³C NMR 193.87, 167.22, 157.02, 149.89, 135.08, 130.59, 130.50, 129.07, 124.50, 122.68, 120.94, 120.85, 117.42, 109.79, 38.80, 12.34.

### (Z)-3-Ethyl-5-[5-(3-fluorophenyl)-2-furyl]methylene-4-oxothiazolidine-2-thione, 1g, R¹═H, R²═F, LJ007, 77%

¹H NMR 7.57 (1H, d, J=8.0 Hz), 7.44 (3H, m), 7.10 (1H, td, J=8.0, 2.0 Hz), 6.94 (1H, d, J=3.6 Hz), 6.87 (1H, d, J=3.6 Hz), 4.20 (2H, q, J=7.2 Hz), 1.32 (3H, t, J=7.2 Hz); ¹³C NMR 193.95, 167.26, 162.60 (d, J=240 Hz), 157.32, 149.83, 130.93, 120.97, 120.84, 120.41, 117.48, 116.20, 115.98, 111.57, 111.33, 109.78, 39.80, 12.33.

### (Z)-3-Ethyl-5-[5-(2-fluorophenyl)-2-furyl]methylene-4-oxothiazolidine-2-thione, 1 h, R¹═F, R²═H, LJ008, 80%

¹H NMR 7.93 (1H, m), 7.47 (1H, s), 7.34 (2H, m), 7.16 (1H, m), 7.02 (1H, d, J=3.6 Hz), 6.95 (1H, d, J=3.6 Hz), 4.21 (2H, q, J=7.2 Hz), 1.29 (3H, t, J=7.2 Hz); ¹³C NMR 194.09, 167.26, 159.20 (d, J=240 Hz), 153.04, 149.12, 130.30, 126.69, 125.00, 121.38, 120.65, 117.56, 116.29, 116.08, 113.90, 113.77, 39.79, 12.34.

### (Z)-3-Ethyl-5-[5-(2-chlorophenyl)-2-furyl]methylene-4-oxothiazolidine-2-thione, 1i, R¹═Cl, R²═H, LJ009, 76%

¹H NMR 7.97 (1H, d, J=8.0 Hz), 7.45 (3H, m), 7.36 (1H, d, J=3.6 Hz), 7.32 (1H, m), 7.29 (1H, m), 6.96 (1H, d, J=3.6 Hz), 4.20 (2H, q, J=7.2 Hz), 1.29 (3H, t, J=7.2 Hz); ¹³C NMR 194.03, 167.26, 154.95, 149.14, 131.09, 130.88, 129.64, 128.29, 127.54, 120.98, 120.91, 117.54, 114.61, 39.80, 12.34.

### (Z)-3-Ethyl-5-[5-(2-methoxyphenyl)-2-furyl]methylene-4-oxothiazolidine-2-thione, 1j, R¹═OMe, R²═H, LJ010, 71%

¹H NMR 7.97 (1H, d, J=8.0 Hz), 7.48 (1H, s), 7.37 (1H, m), 7.16 (2H, m), 7.00 (1H, d, J=8.0 Hz), 6.96 (1H, d, J=3.6 Hz), 4.20 (2H, q, J=7.2 Hz), 3.97 (3H, s), 1.29 (3H, t, J=7.2 Hz); ¹³C NMR 194.35, 167.37, 156.42, 155.88, 148.32, 130.16, 126.90, 121.96, 121.34, 119.40, 118.04, 117.96, 113.99, 111.12, 55.49, 39.74, 12.34.

### (Z)-3-Ethyl-5-[5-(3-methoxyphenyl)-2-furyl]methylene-4-oxothiazolidine-2-thione, 1k, R¹═H, R²═OMe, LJ011, 87%

¹H NMR 7.39 (1H, s), 7.30 (2H, m), 7.26 (1H, d, J=3.6 Hz), 6.94 (2H, m), 6.85 (1H, d, J=3.6 Hz), 4.20 (2H, q, J=7.2 Hz), 3.97 (3H, s), 1.29 (3H, t, J=7.2 Hz); ¹³C NMR 194.13, 167.33, 160.05, 158.62, 149.44, 130.28, 130.20, 121.21, 120.22, 117.73, 117.32, 114.97, 110.05, 109.19, 55.40, 39.75, 12.33 (one carbon unresolved).

### (Z)-3-Ethyl-5-[5-(2-trifluoromethylphenyl)-2-furyl]methylene-4-oxothiazolidine-2-thione, 1l, R¹═CF₃, R²═H, LJ012, 80%

¹H NMR 7.94 (1H, d, J=8.0 Hz), 7.80 (1H, d, J=8.0 Hz), 7.71 (1H, t, J=7.6 Hz), 7.53 (1H, t, J=7.6 Hz), 7.50 (1H, s), 6.95 (2H, s), 4.20 (2H, q, J=7.2 Hz), 1.29 (3H, t, J=7.2 Hz); ¹³C NMR 194.07, 167.31, 154.68, 150.09, 132.35, 129.77, 129.05, 127.00, 126.94, 121.30, 120.73, 117.63, 114.20, 114.15. 39.78, 12.31 (one carbon unresolved).

### (Z)-3-Ethyl-5-((5-(2-hydroxyphenyl)furan-2-yl)methylene)-2-thioxothiazolidin-4-one, 1p, R¹═OH, R²═H, LJ016, 70%

The phenol 1p was prepared from the methyl ether 1j by demethylation with BBr₃ in dry dichloromethane.

¹H NMR 10.60 (1H, s), 7.79 (1H, d, J=8.0 Hz), 7.71 (1H, s), 7.39 (1H, d, J=3.5 Hz), 7.27 (1H, m), 7.07 (1H, m), 4.09 (2H, q, J=7.2H), 1.22 (3H, t, J=7.2 Hz); ¹³C NMR 194.34, 166.70, 156.18, 155.25, 148.18, 130.68, 125.94, 123.85, 120.11, 118.74, 118.15, 116.78, 115.96, 113.96, 39.52, 12.32.

### (Z)-5-((5-(2-Aminophenyl)furan-2-yl)methylene)-3-ethyl-2-thioxothiazolidin-4-one, 1q, R¹═NH₂, R²═H, LJ017, 90%

The aniline 1q was prepared from the nitroarene 1o via reduction with iron in acidic conditions.

¹H NMR 7.58 (1H, dd, J=1.2, 8.0 Hz), 7.49 (1H, s), 7.20 (1H, m), 6.98 (1H, d, J=3.6 Hz), 6.88-6.78 (3H, m), 4.18 (2H, q, J=7.2H), 1.22 (3H, t, J=7.2 Hz); ¹³C NMR 193.71, 166.78, 158.51, 148.32, 146.24, 130.77, 127.48, 123.88, 118.93, 117.76, 117.41, 117.38, 113.02, 111.46, 39.55, 12.44.

### (Z)-3-ethyl-5-((5-phenylthiophen-2-yl)methylene)-2-thioxothiazolidin-4-one, 1r, LJ018, 83%

The same procedure as that for the preparation of compound 1b was used but 5-phenyl-2-thiophenecarboxaldehyde was used instead of 5-phenyl-2-furaldehyde.

¹H NMR 7.87 (1H, s), 7.67 (2H, m), 7.42 (5H, m), 4.19 (2H, q, J=7.2H), 1.32 (3H, t, J=7.2 Hz); ¹³C NMR 192.15, 167.37, 152.46, 137.16, 135.33, 133.07, 129.24, 129.11, 126.06, 125.34, 124.75, 120.71, 39.94, 12.30.

### (Z)-5-(Biphenyl-3-ylmethylene)-3-ethyl-2-thioxothiazolidin-4-one, 1s, LJ019, 67%

The same procedure as that for the preparation of compound 1b was used but biphenyl-3 carboxaldehyde was used instead of 5-phenyl-2-furaldehyde.

¹H NMR 7.81 (1H, s), 7.71-7.41 (9H, m), 4.21 (2H, q, J=7.2H), 1.32 (3H, t, J=7.2 Hz); ¹³C NMR 193.23, 167.57, 142.40, 139.75, 133.80, 132.77, 129.69, 129.35, 128.98, 128.95, 127.92, 127.04, 123.48.

### (Z)-3-Ethyl-5-[5-(2-diazoniophenyl)-2-furyl]methylene-4-oxothiazolidine-2-thione tetrafluoroborate, 1t, LJ020

This compound was made by treatment of the aniline 1q with sodium nitrite and fluoroboric acid.

### (Z)-3-(4-Oxo-5-((5-phenylfuran-2-yl)methylene)-2-thioxothiazolidin-3-yl)propyl acetate, 1u, LJ021, 72%

The same procedure as that for the preparation of compound 1b was used but 3-(3-(tert-butyldimethylsilyloxy)propyl)-2-thioxothiazolidin-4-one was used instead of 3-ethylrhodanine.

¹H NMR 7.79 (2H, m), 7.46 (3H, m), 7.41 (1H, m), 6.96 (1H, d, J=3.6 Hz), 6.88 (1H, d, J=3.6 Hz), 4.25 (2H, t, J=6.7 Hz), 4.13 (2H, t, J=6.8 Hz), 2.10 (2H, m), 2.00 (3H, s); ¹³C NMR 194.41, 170.98, 167.49, 159.02, 149.38, 129.34, 129.16, 128.91, 124.76, 121.61, 119.62, 118.05, 108.99, 61.93, 41.79, 26.33, 20.93.

### (Z)-3-(3-Hydroxypropyl)-5-((5-phenylfuran-2-yl)methylene)-2-thioxothiazolidin-4-one, 1v, LJ022, 73%

The alcohol 1v was prepared from the silyl ether 1u by treatment of 1M HCl in ether.

¹H NMR 7.80 (2H, d, J=7.5 Hz), 7.48 (3H, m), 7.40 (1H, m), 6.99 (1H, d, J=3.6 Hz), 6.88 (1H, d, J=3.6 Hz), 4.32 (2H, t, J=6.0 Hz), 3.61 (2H, m), 2.50 (1H, t, J=6.5 Hz), 1.98 (2H, m); ¹³C NMR 194.81, 168.18, 159.13, 149.22, 129.32, 129.07, 128.74, 124.69, 121.87, 119.25, 118.38, 208.97, 58.73, 41.03, 30.03.

### (Z)-3-Ethyl-5-((2-phenyloxazol-5-yl)methylene)-2-thioxothiazolidin-4-one, 1w, LJ023, 85%

The same procedure as that for the preparation of compound 1b was used but 5-phenyloxazole-2-carboxaldehyde was used instead of 5-phenyl-2-furaldehyde.

¹H NMR 8.14 (2H, m), 7.60 (1H, s), 7.55 (4H, m), 4.21 (2H, q, J=7.2 Hz), 1.29 (3H, t, J=7.2 Hz); ¹³C NMR 193.18, 167.07, 164.48, 147.08, 135.03, 131.75, 129.09, 127.06, 125.87, 123.58, 114.51, 39.94, 12.43.

### (Z)-3-(4-Oxo-5-((5-phenylfuran-2-yl)methylene)-2-thioxothiazolidin-3-yl)propyl)5 (2-oxohexahydro-1H-thieno[3,4-d]imidazol-4-yl)pentanoate, 1x, LJ024, 41%

The biotinylated compound 1x was prepared by the condensation of the alcohol 1v with biotin in the presence of EDC.

¹H NMR 7.79 (2H, d, J=8.0 Hz), 7.54 (1H, s), 7.48 (2H, m), 7.40 (1H, m), 6.99 (1H, d, J=3.6 Hz), 6.88 (1H, d, J=3.6 Hz), 5.63 (1H, s), 5.18 (1H, s), 4.48 (1H, m), 4.28 (3H, m), 4.14 (2H, m), 3.15 (1H, m), 2.92 (1H, dd, J=5.0, 12.5 Hz), 2.72 (1H, d, J=12.5 Hz), 2.34 (2H, m), 2.09 (2H, m), 1.68 (4H, m), 1.44 (2H, m); ¹³C NMR 194.32, 173.36, 167.46, 163.25, 158.91, 149.31, 129.05, 128.96, 128.79, 124.64, 121.77, 119.32, 118.28, 108.96, 61.76, 61.73, 59.99, 55.18, 41.74, 40.49, 33.77, 29.59, 28.17, 28.06, 26.81.

### (Z)-3-Allyl-5-((5-phenylfuran-2-yl)methylene)thiazolidine-2,4-dione, 1y, LJ025, 71%

The same procedure as that for the preparation of compound 1b was used but 3 alkyl-1,3-thiazolidine-2,4-dione was used instead of 3-ethylrhodanine.

¹H NMR 7.76 (dd, J=8.4, 1.2 Hz, 2H), 7.44 (m, 2H), 7.65 (s, 1H), 7.39 (m, 1H), 6.89 (d, J=4.0 Hz, 1H), 6.84 (d, J=4.0 Hz, 1H), 5.88 (m, 1H), 5.27 (m, 2H), 4.36 (dt, J=5.5, 1.5 Hz, 2H), ¹³C NMR 168.61, 165.77, 158.11, 149.02, 130.39, 129.11, 129.07, 124.59, 120.38, 119.26, 118.73, 118.26, 108.49, 43.69.

### 3-Allyl-5-((5-phenylfuran-2-yl)methyl)-4-oxothiazolidin-2-thione, LJ033, 77%

This saturated compound was made by reduction of the C—C double bond of LJ001 with sodium borohydride in ethyl acetate.

¹H NMR 7.66 (dd, J=8.4, 1.2 Hz, 2H), 7.44 (t, J=7.2 Hz, 2H), 7.31 (t, J=7.5 Hz, 1H), 6.60 (d, J=4.0 Hz, 1H), 6.28 (d, J=4.0 Hz, 1H), 5.81 (m, 1H), 5.24 (m, 2H), 4.65 (d, J=7.2 Hz, 2H), 4.60 (dd, J=9.5, 4.0 Hz, 1H), 3.68 (dd, J=15.0, 4.0 Hz, 1H), 3.33 (q, J=9.5 Hz, 1H); ¹³C NMR 199.74, 174.98, 153.86, 149.03, 130.28, 129.26, 128.58, 127.39, 123.53, 119.29, 110.25, 105.63, 49.80, 46.42, 31.30.

### (Z)—N-(3-(4-Oxo-5-((5-phenylfuran-2-yl)methylene)-2-thioxothiazolidin-3-yl)propyl)acetamide, LJ034, 60%

¹H NMR 7.76 (d, J=5.0 Hz, 2H), 7.65 (s, 1H), 7.47 (t, J=7.5 Hz, 2H), 7.38 (t, J=7.5 Hz, 1H), 6.92 (d, J=4.0 Hz, 1H), 6.86 (d, J=4.0 Hz, 1H), 3.85 (t, J=6.5 Hz, 2H), 3.24 (q, J=6.5 Hz, 2H), 2.01 (s, 3H), 1.87 (m, 2H); ¹³C NMR 170.12, 168.40, 166.47, 158.25, 148.77, 129.07, 128.95, 128.91, 124.52, 120.72, 119.53, 117.65, 108.49, 38.74, 35.83, 27.56, 23.33.

### (Z)-3-(3-Aminopropyl)-5-((5-phenylfuran-2-yl)methylene)-4-oxothiazolidin-2-thione, LJ035, 78%

This amine was made by deprotection of the t-Boc group from LJ036 with 4M HCl in dioxane.

¹H NMR 7.78 (dd, J=8.5, 1.0 Hz, 2H), 7.48 (s, 1H), 7.44 (m, 2H), 7.35 (m, 1H), 6.82 (d, J=4.0 Hz, 1H), 6.78 (d, J=4.0 Hz, 1H), 3.80 (t, J=6.5 Hz, 2H), 3.64 (t, J=6.5 Hz, 2H), 1.99 (m, 6.5 2H); ¹³C NMR 165.28, 156.84, 149.46, 129.28, 128.86, 128.49, 124.28, 118.97, 118.03, 115.98, 108.05, 46.46, 40.41, 19.52.

### (Z)-tert-Butyl 3-(4-oxo-5-((5-phenylfuran-2-yl)methylene)-2-thioxothiazolidine-3-yl)propyl carbamate, LJ036, 50%

The carbamate was made by the condensation reaction of tert-butyl 3-(4-oxo-2-thioxothiazolidin-3-yl)propyl carbamate with 5-phenylfuran-2-carboxaldehyde in ethanol and piperidine.

¹H NMR 7.78 (d, J=7.5 Hz, 2H), 7.49 (m, 3H), 7.40 (m, 1H), 6.96 (d, J=4.0 Hz, 1H), 6.86 (d, J=4.0 Hz, 1H), 5.04 (b, 1H), 4.20 (t, J=6.5 Hz, 2H), 3.15 (t, J=6.5 Hz, 2H), 1.92 (m, 2H), 1.42, (s, 9H); ¹³C NMR 194.47, 167.72, 158.97, 155.80, 149.24, 129.25, 129.05, 128.76, 124.65, 121.63, 119.40, 118.09, 108.91, 41.82, 37.29, 28.33, 27.50.

### (Z)-3-(2,4-Dioxo-5-((5-phenylfuran-2-yl)methylene)thioxothiazolidin-3-yl) propyl-5-(2-oxohexahydro-1H-thieno[3,4-d]imidazol-4-yl)pentanoate, LJ037, 60%

The same procedure as that used for the preparation of compound LJ024 was used for this biotinylated compound LJ037 as well.

¹H NMR 7.80 (dd, J=7.5, 1.0 Hz, 2H), 7.70 (s, 1H), 7.47 (t, J=7.5 Hz, 2H), 7.37 (t, J=7.5 Hz, 1H), 7.08 (d, J=4.0 Hz, 1H), 7.05 (d, J=4.0 Hz, 1H), 4.40 (m, 1H), 4.18 (m, 1H), 4.11 (t, J=5.5 Hz, 2H), 3.86 (t, J=6.5 Hz, 2H), 3.06 (m, 1H), 2.83 (m, 1H), 2.64 (m, 1H), 2.28 (t, J=7.5 Hz, 2H), 2.00 (m, 2H), 1.65 (m, 3H), 1.52 (m, 1H), 1.33 (q, J=7.5 Hz, 2H); ¹³C NMR 204.67, 172.47, 168.04, 165.59, 157.58, 149.20, 129.14, 129.06, 128.83, 124.31, 120.76, 118.52, 118.32, 108.94, 61.65, 61.19, 59.60, 55.36, 39.97, 38.92, 33.41, 28.22, 28.11, 26.53, 24.64.

### (Z)-3-(2-Propenyl)-5-((5-phenylfuran-2-yl)methylene-2-thioxothiazolidin-4-thione, LJ027

LJ027 was made in 67% yield by the treatment of LJ001 with Lawesson's reagent in refluxing toluene.

¹H NMR 7.80 (m, 3H), 7.48 (td, J=6.5, 1.0 Hz, 2H), 7.40 (m, 1H), 7.08 (d, J=4.0 Hz, 1H), 6.90 (d, J=4.0 Hz, 1H), 5.89 (m, 1H), 5.31 (m, 2H), 5.23 (m, 2H), ¹³C NMR 196.23, 191.90, 159.07, 150.61, 129.49, 129.13, 129.07, 128.94, 128.68, 124.89, 123.50, 120.18, 119.37, 109.69, 50.53.

### (Z)-3-(2-Propenyl)-5-((5-(2-nitrophenyl)furan-2-yl)methylene-2-thioxothiazolidin-4-thione, LJ028

LJ028 was made in 71% yield by the treatment of LJ015 with Lawesson's reagent in refluxing toluene.

¹H NMR 7.88 (dd, J=8.0, 1.0 Hz, 1H), 7.80 (d, J=8.0 Hz, 1H), 7.75 (s, 1H), 7.71 (td, J=8.0, 1.0 Hz, 1H), 7.55 (td, J=8.0, 1.0 Hz, 1H), 7.04 (d, J=3.5 Hz, 1H), 6.85 (d, J=3.5 Hz, 1H), 4.63 (q, J=7.0 Hz, 2H), 1.27 (t, J=7.0 Hz, 3H), ¹³C NMR 195.90, 192.11, 152.24, 151.72, 132.44, 130.96, 129.67, 129.03, 124.38, 122.61, 121.85, 119.03, 113.85, 44.19, 11.14.

### Example 7

### Medicinal Chemistry and Structure-Activity Relationship (SAR) Studies

To further improve the efficacy, solubility, and therapeutic index (TI) of the arylmethylidene rhodanine derivatives, we conducted structure-activity relationship (SAR) studies with 26 derivatives of LJ001 (Table 2).

TABLE 2
Structure-activity relationship analyses of the LJ-series compounds.

| SAR-1 | IC₅₀ (μM) | 95% CI | IC₅₀ (μM) | 95% CI |
|---|---|---|---|---|
| LJ-001 | 1.0 | 0.74 to 1.90 | 2.57 | 1.51 to 4.39 |
| LJ-002 | 0.7 | 0.27 to 1.76 | 1.79 | 1.18 to 4.39 |
| LJ-003 | 0.9 | 0.48 to 1.98 | 0.95 | 0.57 to 1.58 |
| LJ-004 | 1.1 | 0.42 to 2.90 | 2.49 | 1.32 to 4.70 |
| LJ-005 | 1.5 | 0.80 to 2.85 | 1.22 | 0.63 to 2.36 |
| LJ-006 | 3.6 | 1.56 to 8.15 | 1.42 | 0.74 to 2.72 |
| LJ-007 | 2.5 | 1.22 to 5.38 | 1.45 | 1.07 to 1.96 |
| LJ-008 | 2.4 | 1.70 to 3.60 | 0.91 | 0.35 to 2.38 |
| LJ-009 | 2.9 | 2.05 to 4.27 | 1.26 | 0.49 to 3.26 |
| LJ-010 | 0.8 | 0.57 to 1.28 | 0.40 | 0.17 to 0.92 |
| LJ-011 | 1.5 | 1.14 to 1.86 | 1.16 | 0.59 to 2.31 |
| LJ-012 | 2.3 | 1.50 to 3.85 | 0.82 | 0.46 to 1.47 |
| LJ-013 | Inactive | N/A | Inactive | No Fit |
| LJ-014 | Inactive | N/A | Inactive | No Fit |
| LJ-015 | 1.3 | 0.90 to 1.98 | 1.03 | 0.51 to 2.07 |
| LJ-016 | 2.2 | 1.05 to 4.40 | 2.35 | 1.16 to 4.77 |
| LJ-017 | 3.4 | 1.15 to 9.84 | 1.96 | 1.17 to 3.27 |
| LJ-018 | 4.1 | 0.95 to 17.80 | 2.65 | 1.87 to 3.76 |
| LJ-019 | Inactive | N/A | Inactive | No Fit |
| LJ-020 | 11.1*** | 0.43 to 286.9 | 1.62 | 0.74 to 3.56 |
| LJ-021 | 0.9 | 0.50 to 1.62 | 1.92 | 1.11 to 3.32 |
| LJ-022 | 0.8 | 0.53 to 1.16 | 1.75 | 1.04 to 2.93 |
| LJ-023 | 2.5 | 0.68 to 9.07 | 2.40 | 1.13 to 5.10 |
| LJ-024 | 1.9 | 0.86 to 3.99 | 1.77 | 0.68 to 4.63 |
| LJ-025 | Inactive | N/A | Inactive | No Fit |
| LJ-026 | — | — | Inactive | No Fit |
| LJ-027 | — | — | 0.90** | 0.36 to 2.25 |
| LJ-028 | — | — | 2.01** | 0.92 to 4.39 |
| LJ-029 | — | — | 4.32* | 0.81 to 2.29 |
| LJ-030 | — | — | 0.49 | 0.26 to 0.95 |
| LJ-031 | — | — | 1.04 | 0.50 to 2.16 |
| LJ-032 | — | — | 1.03 | 0.68 to 1.55 |
| LJ-033 | Inactive | N/A | Inactive | No Fit |
| LJ-034 | — | — | Inactive | No Fit |
| LJ-035 | — | — | Inactive | No Fit |
| LJ-036 | — | — | 0.74** | 0.12 to 4.70 |
| LJ-037 | — | — | Inactive | No Fit |

Compound codes, chemical structures, and IC₅₀ values (on NiV-pVSV infections) are shown. Chemical synthetic methods for each compound are provided below.
C.I. = 95% confidence interval;
**= inactive compounds (estimated IC₅₀ greater than 100 μM),
***= IC₅₀ values have been retested; upon re-testing LJ020 had an IC₅₀ of 1.62 μM, C.I. 0.74 μM to 3.56 μM.
**= fit does not seem to visually match data despite good R² . . . IC₅₀ likely worse value
*= slightly poor fit may indicate worse IC50, but definitely active at higher concentrations

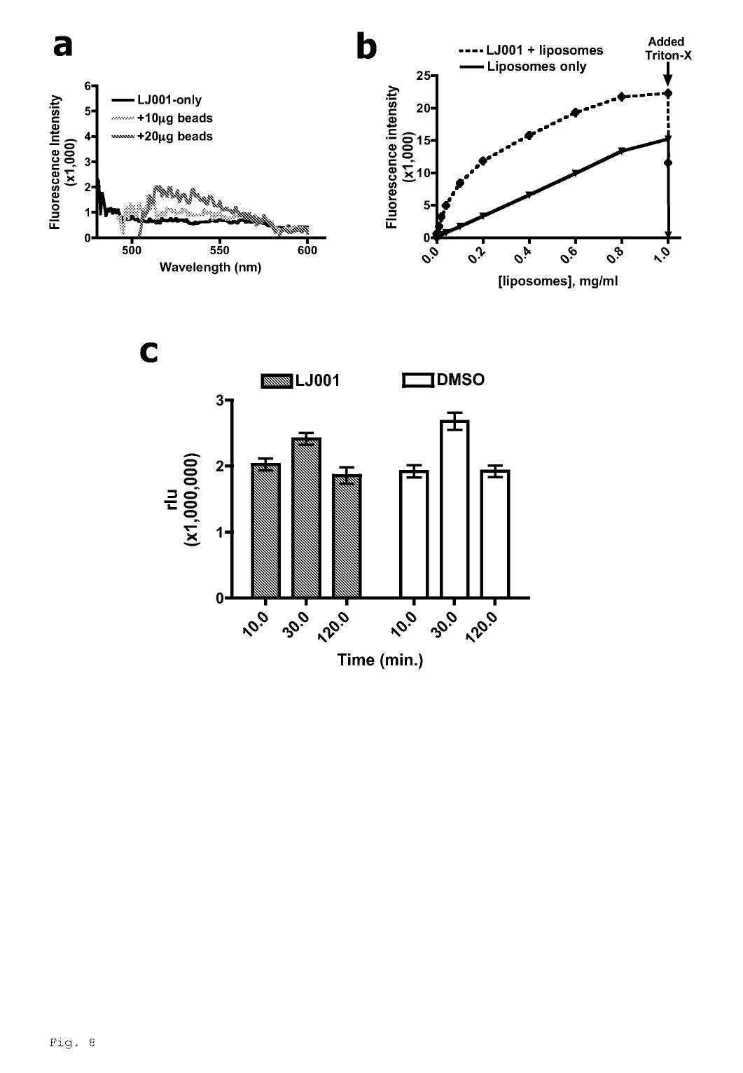

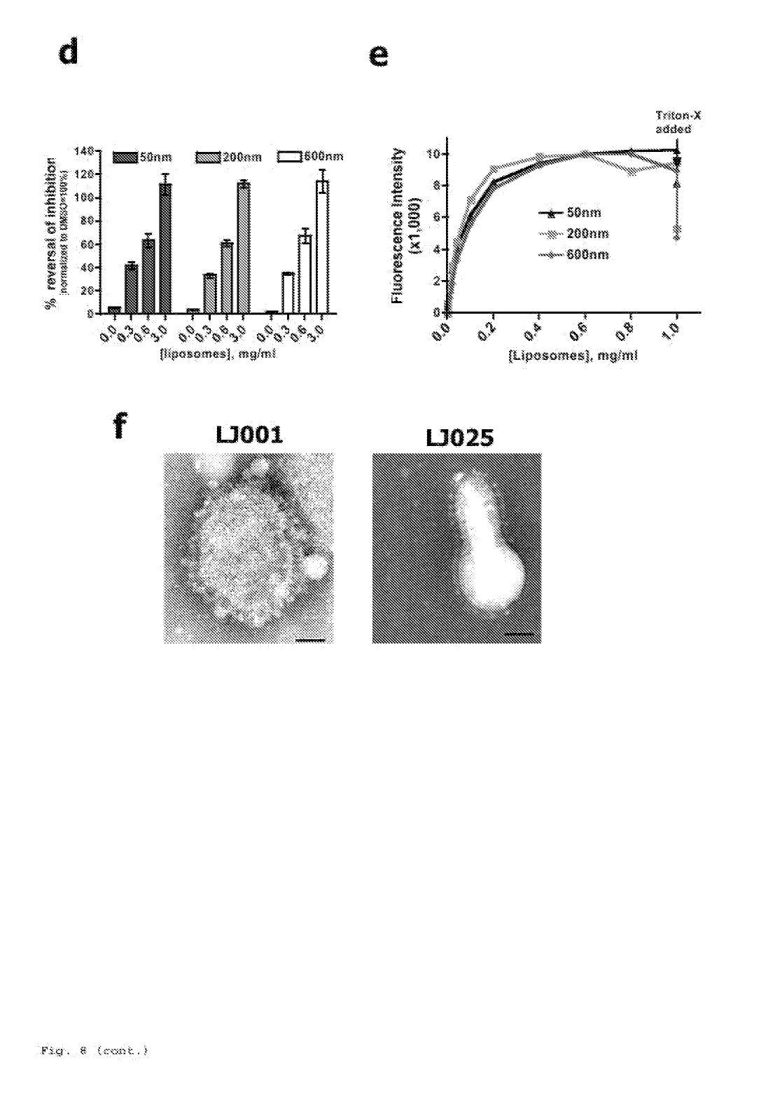

Several analogues showed increased activity in in vitro assays while other analogues with single atom differences (e.g. an oxygen for sulfur replacement, LJ025) were completely inactive both in vitro and in vivo. The arylmethylene rhodanine derivatives comprised generally small alkyl or polar substituents on the ring nitrogen and generally non-polar residues on the aryl ring. Hence, the molecules were generally polar on the left-hand side and non-polar on the right-hand side, as drawn. Small non-polar substituents at the l- and 3-position of the phenyl ring on the right hand side (e.g., halo, methoxy, trifluoromethyl) produced good activity (11006-11012). Small polar substituents, e.g., OH, NH₂, or N₂ (LJ016, LJ017, LJ020), at the 2- and 3-position of the phenyl ring are tolerated but led to generally lower activity than derivatives having non-polar substituents. Derivatives comprising non-polar (LJ001-LJ005) and polar (LJ021-022) groups as substituents on the ring nitrogen (LJ001-005) exhibited good activity. Moreover, a derivative comprising a biotin moiety attached to the ring nitrogen via a 4-atom linker also retained good activity (LJ024). The best aryl group for the arylmethylene in the middle portion of the structure was a 5-substituted 2-furanyl ring. Derivatives comprising a 5-substituted 2-thiophenyl ring (LJ018) or a 2-substituted 5-oxazolyl ring (LJ023) retained activity, albeit at slightly reduced levels, whereas a derivative comprising a 3-substituted phenyl system (LJ019) was inactive. The furyl ring is preferably substituted with a large substituent at the 5-position since methyl and hydrido analogues (LJ013-014) were inactive. Finally, a derivative comprising an oxo function at the polar end of the molecule was completely inactive (LJ025), indicating the importance of the rhodanine thioxo function. The double bond of the arylmethylene unit was also important for activity, since a derivative in which the double bond was reduced (dihydro analogue LJ033) was completely inactive (Table 2 and FIG. 9a).

Thus, arylmethylidene rhodanine derivatives provided herein preferably comprise a thioxo group in the thiazolidine ring and a double bond between the two heterocyclic rings. Without being limited to a particular theory, it is believed that the nonpolar right-hand side of the derivatives may insert into the hydrophobic lipid environment and thereby position the more polar arylmethylene thiazolidine unit for activity (which is much more tolerant of the size and polarity of groups attached at the left-hand side). One possibility is that a nucleophilic species present in the lipid environment of the cell adds to the exomethylene unit to generate a bound intermediate, a process which has precedence in the literature (Carlson et al., Chem Biol, 13:825-37 (2006)). On the other hand, it appears likely that the non-polar (membrane interacting) portion of the derivatives is not wholly responsible for antiviral activity, as LJ025 also intercalated into membranes (FIG. 9b-c) but was otherwise inactive.

### Example 8

### Antiviral Activity In Vivo

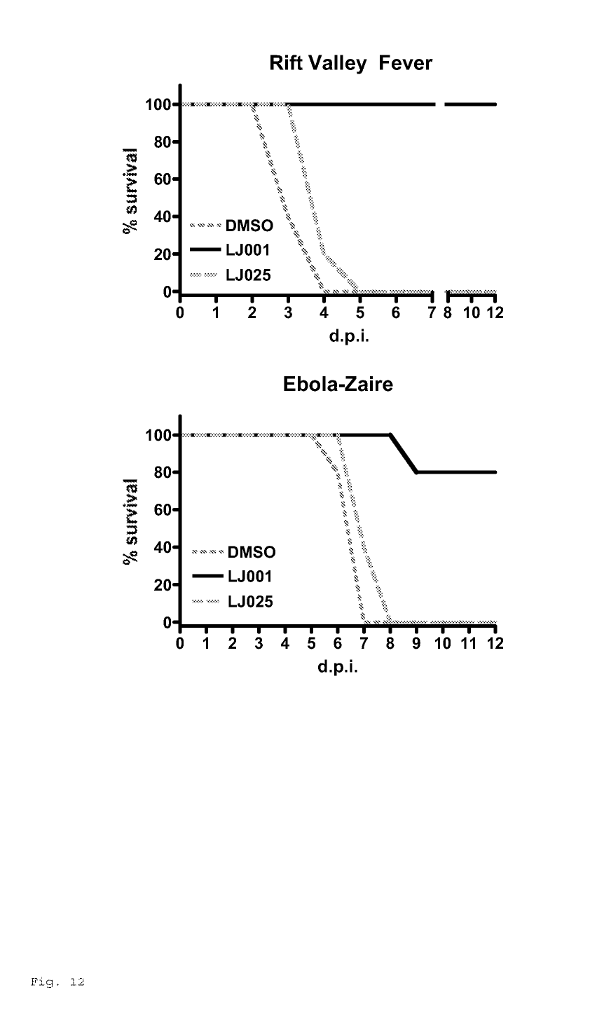

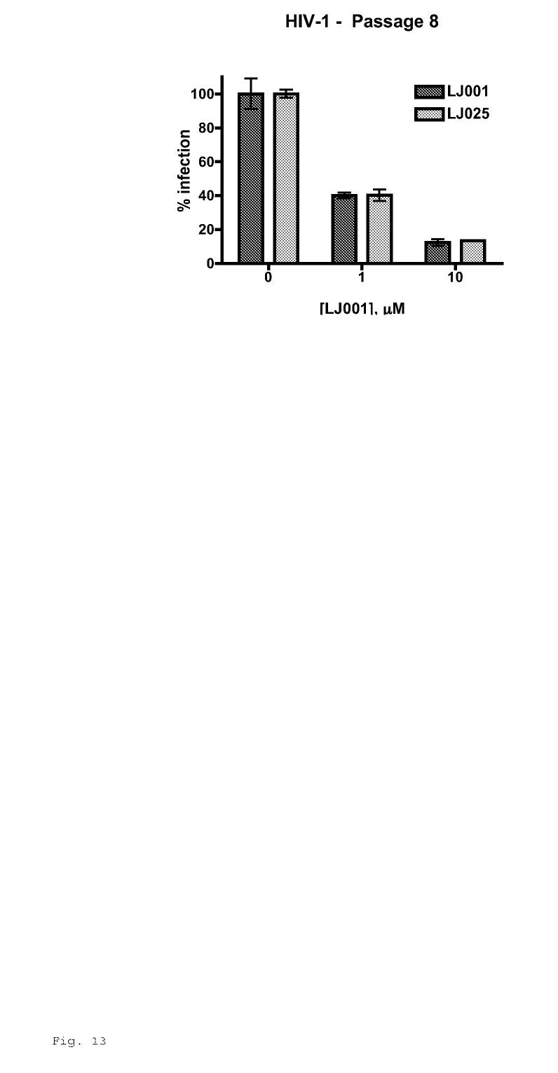

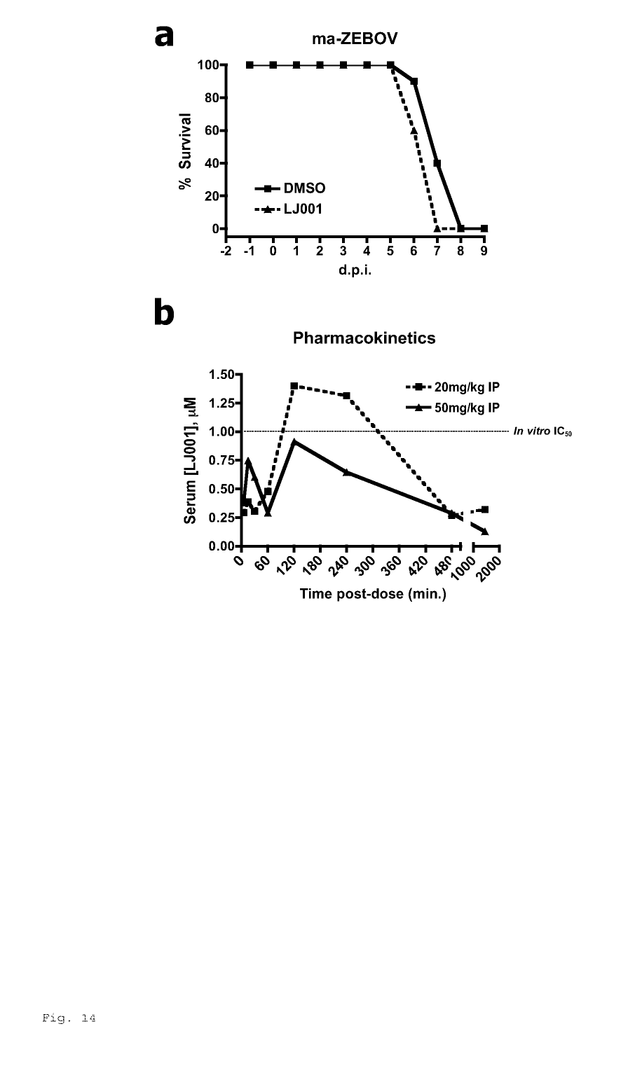

Groups of mice were challenged with lethal doses of RVFV or mouse-adapted Ebola-Zaire virus (maZEBOV) pre-treated (ex vivo) with LJ001, LJ025, or vehicle control (FIG. 12). RVFV and Ebola virus are highly pathogenic viruses classified as NIAID category A priority pathogens, for biodefense purposes, by the US government. Only LJ001 added to RVFV or maZEBOV prior to injection prevented mortality in 100% and 80% (FIG. 12) of animals, respectively. When the maZEBOV challenge experiment was repeated independently, pre-treatment with 10 μM LJ001 protected 100% of the animals (data not shown). The results indicate that LJ001 is capable of preventing virus-induced mortality in vivo. Moreover, after passaging HIV-1 for 4 weeks in sub-neutralizing concentrations of LJ001, there was no evidence of decreased sensitivity to LJ001 (FIG. 13).

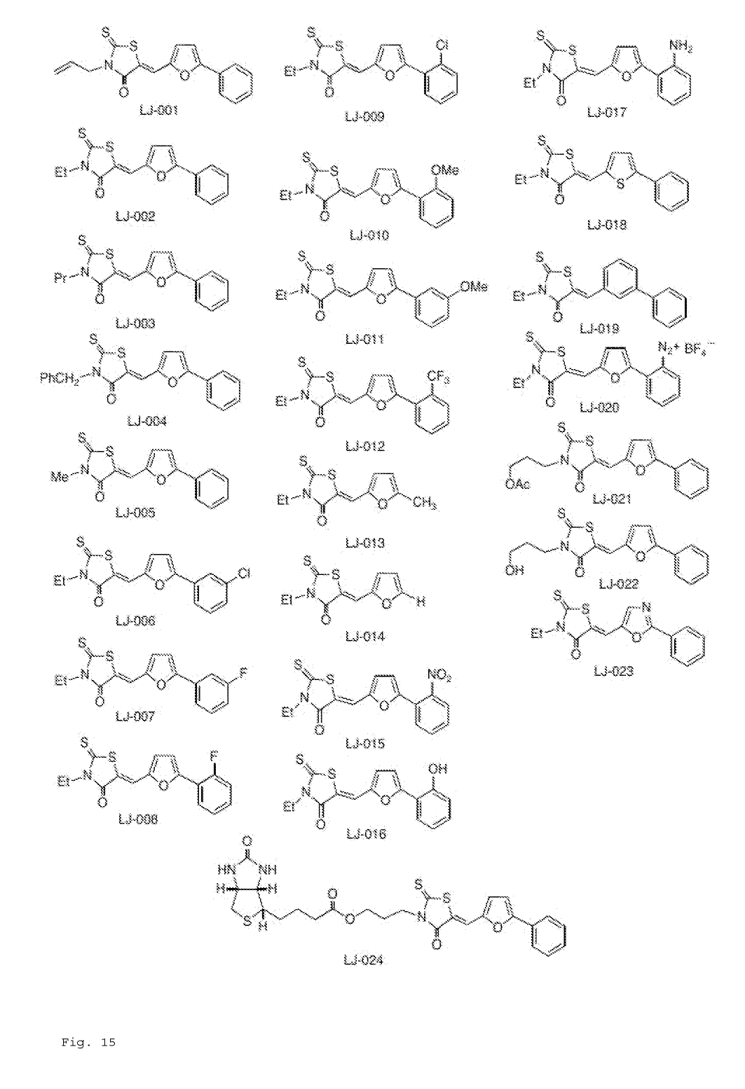

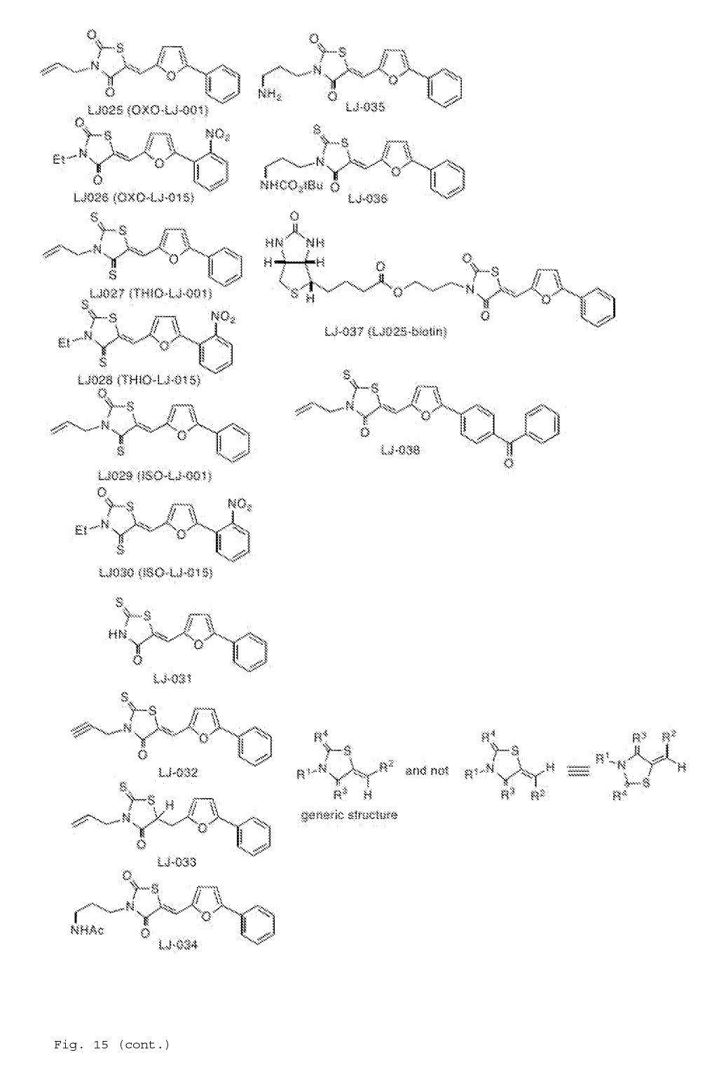

Those skilled in the art will realize that the efficacy of LJ001 and other arylmethylidene rhodanine derivatives provided herein for may depend on formulation and pharmacological considerations as well as the pathogenic profile of the virus and/or other factors. For example, in one experiment, the post-challenge protective efficacy of LJ001 against Ebola was investigated by dosing once daily with LJ001 in 100% DMSO at 50 mg/kg IP after lethal challenge with maEBOV. Although LJ001 did not show efficacy in the post-challenge assay (FIG. 14a), quantification of LJ001 serum levels in subsequent experiments (FIG. 14b) revealed that serum levels of LJ001 did not approach in vitro IC₅₀ concentrations (˜1 μM) until about 2 hours post-IP injection under the conditions of the experiment. Since the biological half-life of LJ001 appears to be about 4 hours, maintaining therapeutic steady-state plasma concentrations of the drug may require a dosing frequency of more than once a day. Moreover, the 20 mg/kg group surprisingly resulted in a higher peak serum concentration than the 50 mg/kg group. Thus, results provided herein indicate that the arylmethylidene rhodanine derivatives have broad ranging antiviral activity. While achieving a level of activity suitable for a particular indication may require optimization of formulation, dose and/or pharmacokinetic parameters, such optimization is well within the purview of those skilled in the art.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference to their entirety.

1. A method of treating or preventing a disease or condition caused by infection with an enveloped virus, the method comprising administering to a subject a therapeutically effective amount of a compound of formula I or a pharmaceutically acceptable salt, prodrug, or derivative thereof, 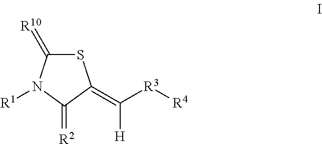
R¹ is H; C₁-C₆ alkyl, C₂-C₆ alkenyl, C₂-C₆ alkynyl, C₁-C₆ alkoxy, C₃-C₆ aryl, C₃-C₆ heteroaryl, C₃-C₆ cycloalkyl, or C₃-C₆ heterocycloalkyl, each optionally substituted with halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷; halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷;
R² is O or S;
R³ is C₅ heteroaryl;
R⁴ is C₅-C₆ aryl or C₅-C₆ heteroaryl, each optionally substituted with C₁-C₆ alkyl, C₂-C₆ alkenyl, C₂-C₆ alkynyl, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷;
R⁵, R⁶, and R⁷ are independently H, C₁-C₆ alkyl, C₂-C₆ alkenyl, C₂-C₆ alkynyl, C₁-C₆ alkoxy, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷; and
R¹⁰ is O or S when R² is S, and R¹⁰ is S when R² is O.

2. The method of claim 1, wherein R² is O.

3. The method of claim 1, wherein R² is S.

4. The method of claim 1, wherein R³ is pyrrolyl, thienyl, furanyl, pyrazolyl, imidazolyl, thiazolyl, isothiazolyl, oxazolyl, or isoxazolyl.

5. The method of claim 1, wherein R³ is pyrrolyl, thienyl, or furanyl.

6. The method of claim 1, wherein R⁴ is phenyl, pyranyl, thiopyranyl, pyridyl, pyrimidinyl, pyrazinyl, or pyridazinyl.

7. The method of claim 1, wherein R⁴ is phenyl.

8. The method of claim 1, wherein the compound is comprises the (Z)-isomer substantially free from the (E)-isomer.

9. The method of claim 1, wherein the compound is of the formula II 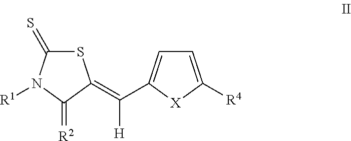
wherein X is O, N, or S.

10. The method of claim 9, wherein X is O and R² is O or S.

11. The method of claim 1, wherein the compound is of the formula V 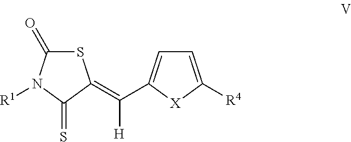
wherein X is O, N, or S.

12. The method of claim 10, wherein X is O.

13. The method of claim 1, wherein the compound is of the formula III 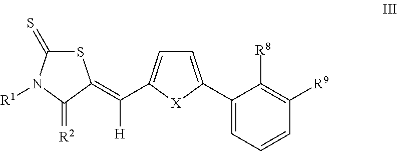
wherein X is O, N, or S; and
R⁸ and R⁹ are independently H, C₁-C₆ alkyl, C₂-C₆ alkenyl, C₂-C₆ alkynyl, C₁-C₆ alkoxy, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷.

14. The method of claim 1, wherein the compound is of the formula VI 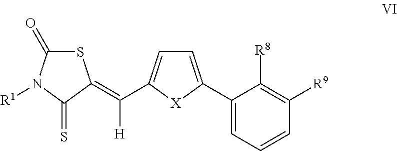
wherein X is O, N, or S; and
R⁸ and R⁹ are independently H, C₁-C₆ alkyl, C₂-C₆ alkenyl, C₂-C₆ alkynyl, C₁-C₆ alkoxy, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷.

15. The method of claim 13, wherein R⁸ and R⁹ are independently H, C₁-C₆ alkyl, —OR⁵, —SR⁵, or —NO₂.

16. The method of claim 14, wherein R⁸ and R⁹ are independently H, C₁-C₆ alkyl, —OR⁵, —SR⁵, or —NO₂.

17. The method of claim 1, wherein the compound is: (Z)-3-allyl-5-(5-phenyl-2-furyl)methylene-4-oxothiazolidine-2-thione (LJ-001); (Z)-3-ethyl-5-(5-phenyl-2-furyl)methylene-4-oxothiazolidine-2-thione (LJ-002); (Z)-3-propyl-5-(5-phenyl-2-furyl)methylene-4-oxothiazolidine-2-thione (LJ-003); (Z)-3-benzyl-5-(5-phenyl-2-furyl)methylene-4-oxothiazolidine-2-thione (LJ-004); (Z)-3-methyl-5-(5-phenyl-2-furyl)methylene-4-oxothiazolidine-2-thione (LJ-005); (Z)-3-Ethyl-5-[5-(3-chlorophenyl)-2-furyl]methylene-4-oxothiazolidine-2-thione (LJ-006); (Z)-3-Ethyl-5-[5-(3-fluorophenyl)-2-furyl]methylene-4-oxothiazolidine-2-thione (LJ-007); (Z)-3-Ethyl-5-[5-(2-fluorophenyl)-2-furyl]methylene-4-oxothiazolidine-2-thione (LJ-008); (Z)-3-Ethyl-5-[5-(2-chlorophenyl)-2-furyl]methylene-4-oxothiazolidine-2-thione (LJ-009); (Z)-3-Ethyl-5-[5-(2-methoxyphenyl)-2-furyl]methylene-4-oxothiazolidine-2-thione (LJ-010); (Z)-3-Ethyl-5-[5-(3-methoxyphenyl)-2-furyl]methylene-4-oxothiazolidine-2-thione (LJ-011); (Z)-3-Ethyl-5-[5-(2-trifluoromethylphenyl)-2-furyl]methylene-4-oxothiazolidine-2-thione (LJ-012); (Z)-3-ethyl-5-((5-(2-nitrophenyl)furan-2-yl)methylene)-2-thioxothiazolidin-4-one (LJ-015); (Z)-3-Ethyl-5-((5-(2-hydroxyphenyl)furan-2-yl)methylene)-2-thioxothiazolidin-4-one (LJ-016); (Z)-5-((5-(2-Aminophenyl)furan-2-yl)methylene)-3-ethyl-2-thioxothiazolidin-4-one (LJ-017); (Z)-3-ethyl-5-((5-phenylthiophen-2-yl)methylene)-2-thioxothiazolidin-4-one (LJ-018); (Z)-3-(4-Oxo-5-((5-phenylfuran-2-yl)methylene)-2-thioxothiazolidin-3-yl)propyl acetate (LJ-021); (Z)-3-(3-Hydroxypropyl)-5-((5-phenylfuran-2-yl)methylene)-2-thioxothiazolidin-4-one (LJ-022); (Z)-3-Ethyl-5-((2-phenyloxazol-5-yl)methylene)-2-thioxothiazolidin-4-one (LJ-023); (Z)-3-(4-Oxo-5-((5-phenylfuran-2-yl)methylene)-2-thioxothiazolidin-3-yl)propyl)-5-(2-oxohexahydro-1H-thieno[3,4-d]imidazol-4-yl)pentanoate (LJ-024); (Z)-3-(2-Propenyl)-5-((5-phenylfuran-2-yl)methylene-2-thioxothiazolidin-4-thione (LJ-027); (Z)-3-ethyl-5-((5-(2-nitrophenyl)furan-2-yl)methylene-2-thioxothiazolidin-4-thione (LJ-028); (Z)-5-(5-phenyl-2-furyl)methylene-4-oxothiazolidine-2-thione (LJ-031); (Z)-3-(2-propynyl)-5-(5-phenyl-2-furyl)methylene-4-oxothiazolidine-2-thione (LJ-032); (Z)—N-(3-(4-Oxo-5-((5-phenylfuran-2-yl)methylene)-2-thioxothiazolidin-3-yl)propyl)acetamide (LJ-034); (Z)-3-(3-Aminopropyl)-5-((5-phenylfuran-2-yl)methylene)-4-oxothiazolidin-2-thione (LJ-035); (Z)-tert-Butyl 3-(4-oxo-5-((5-phenylfuran-2-yl)methylene)-2-thioxothiazolidine-3-yl)propyl carbamate (LJ-036); or (Z)-3-(2,4-Dioxo-5-((5-phenylfuran-2-yl)methylene)thioxothiazolidin-3-yl) propyl-5-(2-oxohexahydro-1H-thieno[3,4-d]imidazol-4-yl)pentanoate (LJ-037).

18. The method of claim 1, wherein the compound is: (Z)-3-allyl-5-(5-phenyl-2-furyl)methylene-2-oxothiazolidine-4-thione (LJ-029); (Z)-3-ethyl-5-((5-(2-nitrophenyl)furan-2-yl)methylene)-2-oxothiazolidin-4-thione (LJ-030); (Z)-3-ethyl-5-(5-phenyl-2 furyl)methylene-2-oxothiazolidine-4-thione (LJ-038); (Z)-3-propyl-5-(5-phenyl-2-furyl)methylene-2-oxothiazolidine-4-thione (LJ-039); (Z)-3-benzyl-5-(5-phenyl-2-furyl)methylene-2-oxothiazolidine-4-thione (LJ-040); (Z)-3-methyl-5-(5-phenyl-2-furyl)methylene-2-oxothiazolidine-4-thione (LJ-041); (Z)-3-Ethyl-5-[5-(3-chlorophenyl)-2-furyl]methylene-2-oxothiazolidine-4-thione (LJ-042); (Z)-3-Ethyl-5-[5-(3-fluorophenyl)-2-furyl]methylene-2-oxothiazolidine-4-thione (LJ-043); (Z)-3-Ethyl-5-[5-(2-fluorophenyl)-2-furyl]methylene-2-oxothiazolidine-4-thione (LJ-044); (Z)-3-Ethyl-5-[5-(2-chlorophenyl)-2-furyl]methylene-2-oxothiazolidine-4-thione (LJ-045); (Z)-3-Ethyl-5-[5-(2-methoxyphenyl)-2-furyl]methylene-2-oxothiazolidine-4-thione (LJ-046); (Z)-3-Ethyl-5-[5-(3-methoxyphenyl)-2-furyl]methylene-2-oxothiazolidine-4-thione (LJ-047); (Z)-3-Ethyl-5-[5-(2-trifluoromethylphenyl)-2-furyl]methylene-2-oxothiazolidine-4-thione (LJ-048); (Z)-3-Ethyl-5-((5-(2-hydroxyphenyl)furan-2-yl)methylene)-2-oxothiazolidin-4-thione (LJ-049); (Z)-5-((5-(2-Aminophenyl)furan-2-yl)methylene)-3-ethyl-2-oxothiazolidin-4-thione (LJ-050); (Z)-3-ethyl-5-((5-phenylthiophen-2-yl)methylene)-2-oxothiazolidin-4-thione (LJ-051); (Z)-3-(4-Thio-5-((5-phenylfuran-2-yl)methylene)-2-oxothiazolidin-3-yl)propyl acetate (LJ-052); (Z)-3-(3-Hydroxypropyl)-5-((5-phenylfuran-2-yl)methylene)-2-oxothiazolidin-4-thione (LJ-053); (Z)-3-Ethyl-5-((2-phenyloxazol-5-yl)methylene)-2-oxothiazolidin-4-thione (LJ-054); (Z)-3-(2-Oxo-5-((5-phenylfuran-2-yl)methylene)-4-thioxothiazolidin-3-yl)propyl)-5-(2-oxohexahydro-1H-thieno[3,4-d]imidazol-4-yl)pentanoate (LJ-055); (Z)-5-(5-phenyl-2-furyl)methylene-2-oxothiazolidine-4-thione (LJ-056); (Z)-3-(2-propynyl)-5-(5-phenyl-2-furyl)methylene-2-oxothiazolidine-4-thione (LJ-057); (Z)—N-(3-(2-Oxo-5-((5-phenylfuran-2-yl)methylene)-4-thioxothiazolidin-3-yl)propyl)acetamide (LJ-058); (Z)-3-(3-Aminopropyl)-5-((5-phenylfuran-2-yl)methylene)-2-oxothiazolidin-4-thione (LJ-059); or (Z)-tert-Butyl 3-(2-oxo-5-((5-phenylfuran-2-yl)methylene)-4-thioxothiazolidine-3-yl)propyl carbamate (LJ-060).

19. The method of claim 1, wherein the virus is hepatitis C virus (HCV), human immunodeficiency virus (HIV), herpes simplex virus type 1 (HSV-1), herpes simplex virus type 2 (HSV-2), Ebola virus, Influenza virus, Nipah virus, Yellow Fever Virus, Dengue virus, Rift Valley Fever Virus, West Nile Virus.

20. The method of claim 1, wherein the compound is administered as a liposomal formulation.

21. A method of preventing infection with an enveloped virus, the method comprising administering to the skin of a subject a therapeutically effective amount of a compound of formula VII or a pharmaceutically acceptable salt, prodrug, or derivative thereof, 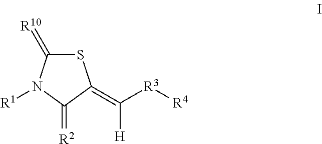
R¹ is H; C₁-C₆ alkyl, C₂-C₆ alkenyl, C₂-C₆ alkynyl, C₁-C₆ alkoxy, C₃-C₆ aryl, C₃-C₆ heteroaryl, C₃-C₆ cycloalkyl, or C₃-C₆ heterocycloalkyl, each optionally substituted with halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷; halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷;
R² is O or S;
R³ is C₅ heteroaryl;
R⁴ is C₅-C₆ aryl or C₅-C₆ heteroaryl, each optionally substituted with C₁-C₆ alkyl, C₂-C₆ alkenyl, C₂-C₆ alkynyl, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷;
R⁵, R⁶, and R⁷ are independently H, C₁-C₆ alkyl, C₂-C₆ alkenyl, C₂-C₆ alkynyl, C₁-C₆ alkoxy, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷; and
R¹⁰ is O or S when R² is S, and R¹⁰ is S when R² is O.

22. The method of claim 21, wherein the compound is comprises the (Z)-isomer substantially free from the (E)-isomer.

23. The method of claim 21, wherein the virus is herpes simplex virus type 1 (HSV-1), herpes simplex virus type 2 (HSV-2), or human immunodeficiency virus (HIV).

24. A compound of formula IV or a pharmaceutically acceptable salt, prodrug, or derivative thereof, 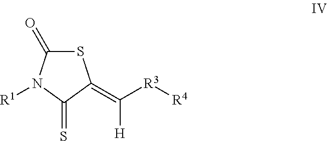
wherein
R¹ is H; C₁-C₆ alkyl, C₂-C₆ alkenyl, C₂-C₆ alkynyl, C₁-C₆ alkoxy, C₃-C₆ aryl, C₃-C₆ heteroaryl, C₃-C₆ cycloalkyl, or C₃-C₆ heterocycloalkyl, each optionally substituted with halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷; halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷;
R³ is C₅ heteroaryl;
R⁴ is C₅-C₆ aryl or C₅-C₆ heteroaryl, each optionally substituted with C₁-C₆ alkyl, C₂-C₆ alkenyl, C₂-C₆ alkynyl, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷; and
R⁵, R⁶, and R⁷ are independently H, C₁-C₆ alkyl, C₂-C₆ alkenyl, C₂-C₆ alkynyl, C₁-C₆ alkoxy, halo, —NO₂, —CF₃, —CN, —OR⁵, —SR⁵, —C(O)R⁵, —NHC(O)R⁵, —C(O)OR⁵, —OC(O)R⁵, —NR⁶R⁷, —C(O)NR⁶R⁷, —NHR⁵C(O)NR⁶R⁷, or —SO₂NR⁶R⁷.

25. The compound of claim 24, wherein the compound is comprises the (Z)-isomer substantially free from the (E)-isomer.

26. The compound of claim 24, which is: (Z)-3-allyl-5-(5-phenyl-2-furyl)methylene-2-oxothiazolidine-4-thione (LJ-029); (Z)-3-ethyl-5-((5-(2-nitrophenyl)furan-2-yl)methylene)-2-oxothiazolidin-4-thione (LJ-030); (Z)-3-ethyl-5-(5-phenyl-2 furyl)methylene-2-oxothiazolidine-4-thione; (Z)-3-propyl-5-(5-phenyl-2-furyl)methylene-2-oxothiazolidine-4-thione; (Z)-3-benzyl-5-(5-phenyl-2-furyl)methylene-2-oxothiazolidine-4-thione; (Z)-3-methyl-5-(5-phenyl-2-furyl)methylene-2-oxothiazolidine-4-thione; (Z)-3-Ethyl-5-[5-(3-chlorophenyl)-2-furyl]methylene-2-oxothiazolidine-4-thione; (Z)-3-Ethyl-5-[5-(3-fluorophenyl)-2-furyl]methylene-2-oxothiazolidine-4-thione; (Z)-3-Ethyl-5-[5-(2-fluorophenyl)-2-furyl]methylene-2-oxothiazolidine-4-thione; (Z)-3-Ethyl-5-[5-(2-chlorophenyl)-2-furyl]methylene-2-oxothiazolidine-4-thione; (Z)-3-Ethyl-5-[5-(2-methoxyphenyl)-2-furyl]methylene-2-oxothiazolidine-4-thione; (Z)-3-Ethyl-5-[5-(3-methoxyphenyl)-2-furyl]methylene-2-oxothiazolidine-4-thione; (Z)-3-Ethyl-5-[5-(2-trifluoromethylphenyl)-2-furyl]methylene-2-oxothiazolidine-4-thione; (Z)-3-Ethyl-5-((5-(2-hydroxyphenyl)furan-2-yl)methylene)-2-oxothiazolidin-4-thione; (Z)-5-((5-(2-Aminophenyl)furan-2-yl)methylene)-3-ethyl-2-oxothiazolidin-4-thione; (Z)-3-ethyl-5-((5-phenylthiophen-2-yl)methylene)-2-oxothiazolidin-4-thione; (Z)-3-(4-Thio-5-((5-phenylfuran-2-yl)methylene)-2-oxothiazolidin-3-yl)propyl acetate; (Z)-3-(3-Hydroxypropyl)-5-((5-phenylfuran-2-yl)methylene)-2-oxothiazolidin-4-thione; (Z)-3-Ethyl-5-((2-phenyloxazol-5-yl)methylene)-2-oxothiazolidin-4-thione; (Z)-3-(2-Oxo-5-((5-phenylfuran-2-yl)methylene)-4-thioxothiazolidin-3-yl)propyl)-5-(2-oxohexahydro-1H-thieno[3,4-d]imidazol-4-yl)pentanoate; (Z)-5-(5-phenyl-2-furyl)methylene-2-oxothiazolidine-4-thione; (Z)-3-(2-propynyl)-5-(5-phenyl-2-furyl)methylene-2-oxothiazolidine-4-thione; (Z)—N-(3-(2-Oxo-5-((5-phenylfuran-2-yl)methylene)-4-thioxothiazolidin-3-yl)propyl)acetamide; (Z)-3-(3-Aminopropyl)-5-((5-phenylfuran-2-yl)methylene)-2-oxothiazolidin-4-thione; or (Z)-tert-Butyl 3-(2-oxo-5-((5-phenylfuran-2-yl)methylene)-4-thioxothiazolidine-3-yl)propyl carbamate.

27. A pharmaceutical composition comprising a compound of claim 1 and at least one pharmaceutically acceptable excipient.

28. The pharmaceutical composition of claim 27, wherein the composition comprises a topical formulation.

29. The pharmaceutical composition of claim 28, which is in the form of a patch, an ointment, a cream, a lotion, a drop, a spray, or an aerosol.

30. The pharmaceutical composition of claim 27, which is in the form of a liposomal formulation.
